(19) United States
(12) Patent Application Publication
(10) Pub. No.: US 2021/0298812 A1
(43) Pub. Date: Sep. 30, 2021

(54) DEVICES AND METHODS FOR VAGINAL TREATMENTS

(71) Applicant: Sofwave Medical Ltd., Yokneam (IL)

(72) Inventors: Ariel SVERDLIK, Tel-Aviv (IL); Shimon ECKHOUSE, Haifa (IL); Assaf GELSTEIN, Haifa (IL)

(73) Assignee: Sofwave Medical Ltd., Yokneam (IL)

(21) Appl. No.: 17/265,218

(22) PCT Filed: Aug. 2, 2019
(86) PCT No.: PCT/IL2019/050878

§ 371 Date: Feb. 2, 2021

Related U.S. Application Data
(60) Provisional application No. 62/713,559, filed on Aug. 2, 2018.

(51) Int. Cl. A61B 18/08; A61B 90/00

(52) U.S. Cl. CPC A61B 18/08; A61B 2018/00994; A61B 90/39

(57) ABSTRACT

A system for delivery of ultrasonic waves to a tissue volume, including:

an ultrasound applicator shaped and sized to be at least partially inserted into a vagina, including:

a body with a non-planar surface, wherein said body is shaped and sized to be at least partly introduced into a vagina;

a plurality of ultrasound transducers distributed on the non-planar surface of the ultrasound applicator body, wherein at least some of the plurality of ultrasound transducers are configured to contact a surface of a wall of the vagina during the generation of ultrasonic waves; at least one cooler configured to apply cooling to prevent overheating of the vagina wall surface being contacted by the transducers;

a control unit, comprising:

a control circuitry electrically connected to the ultrasonic transducers, wherein the control circuitry signals at least some of the ultrasound transducers to generate ultrasonic waves to heat the tissue volume.

### RELATED APPLICATION/S

This application claims the benefit of priority under 35 USC § 119(e) of U.S. Provisional Patent Application No. 62/713,559 filed 2 Aug. 2018, the contents of which are incorporated herein by reference in their entirety.

### FIELD AND BACKGROUND OF THE INVENTION

The present invention, in some embodiments thereof, relates to female urogenital system treatments and, more particularly, but not exclusively, to tissue remodeling treatments in the urogenital system and/or anal incontinence treatments. Laser energy is currently being used for vaginal treatments by affecting superficial tissue with mucosal layers, and is used for treating the Vulva (Labia), Vaginal opening/Introitus and/or the Vaginal canal (up to a distance of 10 cm).

### SUMMARY OF THE INVENTION

Following are some examples of some embodiments of the invention:

Example 1. A system for delivery of ultrasonic waves to a tissue volume, comprising: an ultrasound applicator shaped and sized to be at least partially inserted into a vagina, comprising:

a body with a non-planar surface, wherein said body is shaped and sized to be at least partly introduced into a vagina;

a plurality of ultrasound transducers distributed on said non-planar surface of said ultrasound applicator body, wherein said plurality of ultrasound transducers are configured to contact a surface of a wall of said vagina during the generation of ultrasonic waves; a cooling module configured to apply cooling to prevent overheating of said vagina wall surface being contacted by said transducers;

a control unit, comprising:

a control circuitry electrically connected to said ultrasonic transducers, wherein said control circuitry signals at least some of said ultrasound transducers to generate ultrasonic waves at different angular directions with parameter values sufficient to deliver ultrasonic energy to said tissue volume.

Example 2. A system according to any one of the previous examples, wherein said cooling module is configured to cool said vagina surface being contacted by said transducers through said transducers and/or between said transducers.
Example 3. A system according to any one of the previous examples wherein said control circuitry signals at least some of said ultrasound transducers to simultaneously generate said ultrasonic waves.
Example 4. A system according to any one of the previous examples, wherein said plurality of ultrasound transducers are thin transducers having a thickness in a range of 0.1-2 mm.
Example 5. A system according to example 1, wherein each of said plurality of ultrasound transducers comprise PZT element.
Example 6. A system for delivery of ultrasonic waves to a tissue volume, comprising: an ultrasound applicator having a body with a non-planar surface comprising:

a plurality of ultrasound transducers distributed on said non-planar surface of said ultrasound applicator body, wherein said plurality of ultrasound transducers are configured to contact a surface of a tissue during the generation of ultrasonic waves;

one or more TECs configured to apply cooling to prevent overheating of said tissue surface being contacted by said transducers;

a control unit, comprising:

a control circuitry electrically connected to said ultrasonic transducers, wherein said control circuitry signals at least some of said ultrasound transducers to generate ultrasonic waves at different angular directions with parameter values sufficient to deliver ultrasonic energy to said tissue volume.

Example 7. A system according to example 6, wherein said ultrasound applicator comprises a cooling bath comprising a cooling liquid, and wherein a hot surface of said one or more TECs is shaped and sized to contact a surface of said cooling bath.
Example 8. A system according to any one of examples 6 or 7, wherein said control circuitry signals at least some of said ultrasound transducers to simultaneously generate said ultrasonic waves.
Example 9. A system according to any one of examples 6 to 8, wherein a cold surface of said TEC is configured to cool said tissue surface being contacted by said transducers through said transducers and/or between said transducers.
Example 10. A system according to any one of examples 6 to 9, wherein said plurality of ultrasound transducers are thin transducers.
Example 11. An ultrasound applicator, comprising:

a first surface comprising at least one ultrasound transducer, configured to move towards an opposite second surface and to clamp a tissue between said first surface and said opposite second surface,

wherein said at least one ultrasound transducer emits ultrasonic waves into a tissue volume within said clamped tissue.
Example 12. A method for delivery of ultrasonic waves to a tissue volume, comprising: attaching a surface of an ultrasound applicator to a surface of a vagina wall;
simultaneously emitting ultrasonic waves at different angular directions to a tissue volume located in said vagina wall;
cooling said surface of said vagina wall during said simultaneously emitting.
Example 13. A method for vagina rejuvenation, comprising:
attaching an ultrasound applicator to a surface of a vagina wall;
emitting ultrasonic waves at angular directions larger than 90 degrees to a tissue volume located within said vagina wall;
cooling said vagina wall surface contacting said applicator during said emitting.
Example 14. A method for reshaping the labia, comprising:
attaching a surface of an ultrasound applicator to a surface of a labia wall;
emitting ultrasonic waves to a tissue volume located within said labia wall while applying force on said labia wall;
cooling said labia wall surface during said emitting.
Example 15. A method for treating urinal incontinence, comprising:
attaching an ultrasound applicator to a surface of a vagina wall;
emitting ultrasonic waves with low frequency values to a tissue volume located near the urethra wall;
cooling said vagina wall surface during said emitting.
Example 16. A method according to example 15, wherein said tissue volume is located at a distance of up to 15 mm from the urethra wall.
Example 17. A method according to any one of examples 15 or 16, comprising introducing a catheter into the urethra, and wherein said cooling comprises cooling said urethra wall during said emitting by said catheter.
Example 18. A method for treating tissue layers in a vagina wall, comprising:
attaching an ultrasound applicator to a surface of a vagina wall;
emitting ultrasonic waves with frequency values sufficient to penetrate into a sub mucosa and/or adventitia layers within said vagina wall;
cooling said vagina wall surface during said emitting.
Example 19. A system for delivery of ultrasonic waves to a tissue volume, comprising: an ultrasound applicator shaped and sized to be at least partially inserted into a vagina, comprising:

a body with a non-planar surface;

a plurality of ultrasound transducers axially distributed on said non-planar surface of said ultrasound applicator body, wherein said plurality of ultrasound transducers are configured to contact a surface of a wall of said vagina during the generation of ultrasonic waves; a cooling module configured to apply cooling to prevent overheating of said vagina wall surface being contacted by said transducers;

a control unit, comprising:

a control circuitry electrically connected to said ultrasonic transducers, wherein said control circuitry signals at least some of said ultrasound transducers to generate ultrasonic waves at different angular directions with parameter values sufficient to deliver ultrasonic energy to said tissue volume.

Example 20. A system for delivery of ultrasonic waves to a tissue volume, comprising: an ultrasound applicator shaped and sized to be at least partially inserted into a vagina, comprising:

a body with a non-planar surface;

a plurality of ultrasound transducers axially distributed on said non-planar surface of said ultrasound applicator body, wherein said plurality of ultrasound transducers are configured to contact a surface of a wall of said vagina during the generation of ultrasonic waves;

one or more cooling elements configured to apply cooling to prevent overheating of said vagina wall surface being contacted by said transducers, wherein some of said one or more cooling elements are attached to two or more of said plurality of transducers;

a control unit, comprising:
a control circuitry electrically connected to said ultrasonic transducers, wherein said control circuitry signals at least some of said ultrasound transducers to generate ultrasonic waves at different angular directions with parameter values sufficient to deliver ultrasonic energy to said tissue volume.
   Example 21. A system according to example 20, wherein said one or more cooling elements comprises at least one TEC and/or at least one heat-conducting base.
   Example 22. A system according to example 21, wherein said heat-conducting base is a heat conducting aluminum base.
   Example 23. A method for delivery of ultrasonic waves to a tissue volume, comprising: attaching a surface of an ultrasound applicator to a surface of a vagina wall;
   simultaneously emitting ultrasonic waves at different angular directions to a tissue volume located in said vagina wall;
   cooling said surface of said vagina wall in a timed relationship with said simultaneously emitting.
   Example 24. A method according to example 23, wherein said timed relationship, comprises before and during said simultaneously emitting.
   Example 25. A method according to any one of examples 23 or 24, wherein said timed relationship comprises after said simultaneously emitting.
   Example 26. An ultrasound applicator comprising:

an elongated hollow body having a non-planar surface, wherein said elongated body comprises at least one acoustic window in said non-planar surface;

one or more ultrasound transducers connected to a shaft passing through an inner lumen of said elongated hollow body, wherein said one or more ultrasound transducers are configured to transmit ultrasound waves through said at least one window;

a cooling liquid circulating in said inner lumen between said one or more ultrasound transducers and said at least one window; wherein said circulating cooling liquid reduces temperature levels of said non-planar surface during the activation of said one or more ultrasound transducers.

Following are some additional examples of some embodiments of the invention: Example 1. A system for delivery of ultrasonic waves to a tissue volume, comprising:

an ultrasound applicator shaped and sized to be at least partially inserted into a vagina, comprising:

a body with a non-planar surface, wherein said body is shaped and sized to be at least partly introduced into a vagina;

a plurality of ultrasound transducers distributed on said non-planar surface of said ultrasound applicator body, wherein at least some of said plurality of ultrasound transducers are configured to contact a surface of a wall of said vagina during the generation of ultrasonic waves; at least one cooler configured to apply cooling to prevent overheating of said vagina wall surface being contacted by said transducers;

a control unit, comprising:

a control circuitry electrically connected to said ultrasonic transducers, wherein said control circuitry signals at least some of said ultrasound transducers to generate ultrasonic waves to heat said tissue volume.

Example 2. A system according to example 1, wherein said non-planar surface has a radius of curvature smaller than 5 cm.
Example 3. A system according to any one of the previous examples, wherein said plurality of ultrasound transducers are axially distributed on at least a portion said non-planar surface.
Example 4. A system according to any one of the previous examples, wherein said plurality of transducers are circumferentially distributed on at least a portion of said non-planar surface.
Example 5. A system according to any one of the previous examples wherein said body is a tubular body.
Example 6. A system according to example 5, wherein a portion of said tubular body comprising said ultrasound transducers has a radius smaller than 5 cm.
Example 7. A system according to any one of the previous examples, wherein said control circuitry signals at least some of said ultrasound transducers to generate said ultrasonic waves at different angular directions larger than 90 degrees to heat said tissue volume.
Example 8. A system according to any one of the previous examples, wherein said control circuitry signals at least some of said ultrasound transducers to generate ultrasonic waves with different intensity values and/or different frequency values and/or for different time duration compared to other ultrasound transducers of the applicator.
Example 9. A system according to any one of the previous examples, wherein said control circuitry is electrically connected to said cooler, and wherein said control circuitry signals said cooler to apply said cooling prior to and/or during and/or after signaling said at least some of said ultrasound transducers to generate said ultrasonic waves.
Example 10. A system according to any one of the previous examples, wherein said cooling module is configured to cool said vagina surface being contacted by said transducers through said transducers and/or between said transducers.
Example 11. A system according to any one of the previous examples wherein said control circuitry signals at least some of said ultrasound transducers to simultaneously generate said ultrasonic waves.
Example 12. A system according to any one of the previous examples, comprising a memory, and wherein said control circuitry activates said at least some of said ultrasound transducers according to one or more treatment plans, for different regions and/or depth locations within the vagina, stored in said memory.
Example 13. A system according to example 12, wherein said treatment plans comprise safety intensity and/or frequency and/or activation time values for sais different regions and/or depth locations within the vagina.
Example 14. A system according to any one of the previous examples, comprising a liquid sealed introducer configured to cover an area of said ultrasound transducers of said ultrasound applicator positioned within the vagina and to prevent contact between liquids of the vagina with said ultrasound transducers area.
Example 15. A system according to example 14, wherein said control circuitry signals said cooler to generate and apply said cooling through said liquid sealed introducer to said wall surface.
Example 16. A system according to any one of the previous examples, wherein said plurality of ultrasound transducers are thin transducers having a thickness in a range of 0.1-2 mm.
Example 17. A system according to any one of the previous examples, wherein said body comprises a plurality of visual depth markings on an external surface of said body, wherein said visual depth markings are configured to deliver a visual indication regarding a penetration depth of the ultrasound applicator into said vagina.
Example 18. A system according to any one of the previous examples, comprising a handle coupled to a proximal end of said body, wherein said handle comprises one or more gripping members shaped and sized to allow holding of the handle with a single hand.
Example 19. A system according to example 18, wherein said ultrasound applicator comprises one or more visual rotation orientation markings on said body and/or on said handle configured to deliver a visual indication regarding a rotation of the ultrasound applicator within the vagina.
Example 20. A system according to any one of the previous examples, wherein said ultrasound applicator comprises at least one position sensor configured to sense a position of the applicator within the vagina, electrically connected to said control circuitry, wherein said control circuitry is configured to calculate a position of said ultrasound applicator within the vagina based on signals received from said at least one position sensor.
Example 21. A system according to example 20, wherein said position comprises orientation and/or rotation angle.
Example 22. A system according to example 20, wherein said control circuitry is configured to modify intensity and/or frequency of said ultrasonic waves according to said calculated position and/or according to a relation between said calculated position and a selected target location within the vagina wall.
Example 23. A system according to any one of the previous examples, wherein said ultrasound applicator comprises at least one expander coupled to said body, wherein said expander is configured to expand said body such that said at least some of said plurality of transducers are pushed against said surface of said vagina wall.
Example 24. A system according to any one of the previous examples, wherein said ultrasonic waves generated by said plurality of ultrasound transducers are non converging ultrasonic waves.
Example 25. A system according to any one of the previous examples wherein said ultrasound transducers are flat.
Example 26. A system according to any one of the previous examples, wherein said at least one cooler comprises at least one thermoelectric cooler (TEC) having a hot surface and a cold surface.
Example 27. A system according to example 26, wherein said at least one TEC is placed in contact with one or more of the ultrasound transducers.
Example 28. A system according to example 26, wherein said ultrasound applicator comprises a cooling bath comprising a cooling liquid, and wherein said hot surface of said at least one TEC is shaped and sized to contact a surface of said cooling bath.
Example 29. A system according to any one of examples 26 to 28, wherein said cold surface of said TEC is configured to cool said tissue surface being contacted by said transducers through said transducers and/or between said transducers.
Example 30. An ultrasound applicator, comprising:

a first surface comprising at least one ultrasound transducer and a second opposite surface coupled to said first surface, wherein said first surface and said second opposite surface move towards each other to clamp a tissue between them;

wherein said at least one ultrasound transducer emits ultrasonic waves into a tissue volume within said clamped tissue to heat said tissue volume to a temperature larger than 55° C.
Example 31. An ultrasound applicator according to example 30, comprising a hinge, and wherein said first surface and said second opposite surface are pivotally connected to each other by said hinge.
Example 32. An ultrasound applicator according to any one of examples 30 or 31, comprising at least one pressure sensor, configured to sense a pressure applied on said clamped tissue by said first surface and/or said second surface.
Example 33. A method for delivery of ultrasonic waves to a tissue volume, comprising: contacting a surface of a vagina wall with a surface of an ultrasound applicator; simultaneously emitting ultrasonic waves at different angular directions to a tissue volume located in said vagina wall to heat said tissue volume to a temperature larger than 55° C.; cooling said surface of said vagina wall during said simultaneously emitting.
Example 34. A method according to example 33, comprises axially moving said surface of said ultrasound applicator to a different axial location within the vagina following said simultaneously emitting.
Example 35. A method according to any one of examples 33 or 34, comprising rotating said surface of said ultrasound applicator to a different rotation orientation within said vagina following said simultaneously emitting.
Example 36. A method according to example 34, comprising modifying intensity and/or frequency of said ultrasonic waves based on said different axial location.
Example 37. A method according to any one of examples 33 to 36, comprising positioning said surface of said ultrasound applicator within a liquid sealed introducer prior to said contacting and wherein said cooling comprises cooling said surface of said vagina wall through said liquid sealed introducer.
Example 38. A method according to example 33, wherein said simultaneously emitting comprises simultaneously emitting ultrasonic waves at angular directions larger than 90 degrees to said tissue volume.
Example 39. A method for reshaping the labia, comprising:
contacting a surface of a labia wall with a surface of an ultrasound applicator;
emitting ultrasonic waves to a tissue volume located within said labia wall to heat said tissue volume to a temperature larger than 55° C., while applying force on said labia wall;
cooling said labia wall surface during said emitting.
Example 40. A method according to example 39, wherein said contacting comprises contacting a first surface of said labia wall with said surface of an ultrasound applicator and an opposite surface of said labia wall with a different surface of said ultrasound applicator, wherein said emitting comprises emitting ultrasonic waves from said surface and said different surface of said ultrasound applicator to said tissue volume.
Example 41. A method for treating urinal incontinence, comprising:
contacting a surface of a vaginal wall with a surface of an ultrasound applicator to a surface of a vagina wall;
emitting ultrasonic waves with frequency values lower than 11 MHz to a tissue volume located near the urethra;
cooling said vagina wall surface during said emitting.
Example 42. A method according to example 41, comprising introducing a catheter into the urethra, and wherein said cooling comprises cooling said urethra wall during said emitting by said catheter.
Example 43. A method for treating tissue layers in a vagina wall, comprising:
contacting a vagina wall with an ultrasound applicator;
emitting ultrasonic waves with intensity of 40-60w/cm\hat{ }2 for 1-3 seconds and/or 35-55 W/cm\hat{ }2 for 3-7 seconds to penetrate into a sub mucosa and/or adventitia layers within said vagina wall;
cooling said vagina wall surface during said emitting.
Example 44. A method according to example 43, comprises axially moving said ultrasound applicator to a different axial location within the vagina following said emitting.
Example 45. A method according to any one of examples 43 or 44, comprising rotating said ultrasound applicator to a different rotation orientation within said vagina following said emitting.
Example 46. A system for delivery of ultrasonic waves to a tissue volume, comprising: an ultrasound applicator shaped and sized to be at least partially inserted into a vagina, comprising:

a body with a non-planar surface;

a plurality of ultrasound transducers axially distributed on said non-planar surface of said ultrasound applicator body, wherein said plurality of ultrasound transducers are configured to contact a surface of a wall of said vagina during the generation of ultrasonic waves;

one or more cooling elements configured to apply cooling to prevent overheating of said vagina wall surface being contacted by said transducers, wherein some of said one or more cooling elements are attached to two or more of said plurality of transducers;

a control unit, comprising:

a control circuitry electrically connected to said ultrasonic transducers, wherein said control circuitry signals at least some of said ultrasound transducers to generate ultrasonic waves at different angular directions with parameter values sufficient to deliver ultrasonic energy to said tissue volume.

Example 47. A system according to example 46, wherein said one or more cooling elements comprises at least one TEC and/or at least one heat-conducting base.
Example 48. A system according to example 47, wherein said heat-conducting base is a heat conducting aluminum base.
Example 49. An ultrasound applicator comprising:

an elongated hollow body having a non-planar surface, wherein said elongated body comprises at least one acoustic window in said non-planar surface;

one or more ultrasound transducers connected to a shaft passing through an inner lumen of said elongated hollow body, wherein said one or more ultrasound transducers are configured to transmit ultrasound waves through said at least one window;

a cooling liquid circulating in said inner lumen between said one or more ultrasound transducers and said at least one window; wherein said circulating cooling liquid reduces temperature levels of said non-planar surface during the activation of said one or more ultrasound transducers.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as measure tissue temperature and/or transmit ultrasound energy, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the invention are herein described, by way of example only, with reference to the accompanying drawings. With specific reference now to the drawings and images in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A is an anatomical illustration of a female urogenital system, according to some embodiments of the invention;

FIG. 1B is a flow chart of a general process for treating vaginal tissue, according to some exemplary embodiments of the invention;

FIG. 2 is a block diagram of a system for treating vaginal tissue, according to some embodiments of the invention;

FIG. 3A is an image showing the effect of energy emitted during vaginal treatments on deep tissue layers, according to some embodiments of the invention;

FIG. 3B is a detailed flow chart of a process for treating urogenital system tissue, according to some embodiments of the invention;

FIG. 3C is a detailed flow chart of a system operation process in relation to urogenital system tissue treatment, according to some embodiments of the invention;

FIG. 3D is a schematic illustration showing control of ultrasonic waves delivery in different directions, according to some embodiments of the invention;

FIGS. 4A-4C are schematic illustration of an applicator having a plurality of radially distributed transducers and a single thermoelectric cooler (TEC), according to some embodiments of the invention;

FIG. 4D is a cross-section view of an applicator having a plurality of radially distributed transducers and a single TEC, according to some embodiments of the invention;

FIG. 4E is a cross-section view showing electrical connection of transducers, according to some embodiments of the invention;

FIG. 4F is a schematic illustration showing interaction between a heat-conducting base and transducers, according to some embodiments of the invention;

FIG. 4G is a schematic illustration of a printed circuit board (PCB), according to some embodiments of the invention;

FIG. 4H is a schematic illustration of a heat-conducting base for a plurality of transducers, according to some embodiments of the invention;

FIG. 5A is a schematic illustration of an applicator with a front-facing PCB, according to some embodiments of the invention;

FIG. 5B is a schematic illustration of a front-facing PCB, according to some embodiments of the invention;

FIGS. 6A and 6B are schematic illustrations of an applicator having groups of axially arranged transducers radially distributed on the surface of the applicator, according to some embodiments of the invention;

FIG. 6C is a schematic illustration of a heat-conducting base for a plurality of axially arranged transducers, according to some embodiments of the invention;

FIG. 6D is a cross-section view of the applicator shown in FIGS. 6A and 6B, according to some embodiments of the invention;

FIG. 6E is a schematic illustration of a plurality of axially arranged transducers positioned on top of the heat-conducting base shown in FIG. 6C, according to some embodiments of the invention;

FIG. 6F is a schematic illustration showing the internal arrangement of a cooling system, PCB and transducers in an ultrasound applicator, according to some embodiments of the invention;

FIGS. 7A-7C are schematic illustrations of an applicator with a plurality of axially arranged transducers with 3 TECs, according to some embodiments of the invention;

FIG. 7D is a schematic cross-section of the applicator shown in FIGS. 7A-7C, according to some embodiments of the invention;

FIG. 7E is a schematic illustration of a heat-conducting base adaptor, according to some embodiments of the invention;

FIG. 7F is a schematic illustration of a PCB for electrically connecting axially arranged transducers attached to the heat-conducting base adaptor, according to some embodiments of the invention;

FIG. 7G is a schematic illustration of axially arranged transducers electrically connected to the PCB shown in FIG. 7F, according to some embodiments of the invention;

FIGS. 7H-7J are schematic illustrations showing a geometrical relation between a single TEC with two arrays of axially arranged transducers, according to some embodiments of the invention;

FIG. 7K is a schematic illustration of a cooling bath connected to 1 out of 3 TECs, according to some embodiments of the invention;

FIG. 8A is a schematic illustration of an applicator for delivery of vaginal tissue treatments, according to some embodiments of the invention;

FIGS. 8B-8D are schematic illustrations showing the applicator of FIG. 8A and the Vagina in a timed relationship with a Vagina treatment, according to some embodiments of the invention;

FIG. 9 is a schematic illustration showing the applicator of FIG. 8A and the Vagina during Urethra wall treatment, according to some embodiments of the invention;

FIGS. 10A-10C are schematic illustrations showing the applicator of FIG. 8A when treating Labia minor and/or Labia major tissues, according to some embodiments of the invention;

FIG. 11A is a schematic illustration of an applicator with a plurality of forward facing transducers, according to some embodiments of the invention;

FIGS. 11B and 11C are schematic illustrations of an applicator with forward facing transducers during treatment of major and/or minor labia tissue, according to some embodiments of the invention;

FIG. 12A is a schematic illustration of a tissue holder ultrasound applicator in an open state, according to some embodiments of the invention;

FIG. 12B is a schematic illustration of a tissue holder ultrasound applicator in a closed state, according to some embodiments of the invention;

FIGS. 12C and 12D are schematic illustrations of a tissue holder ultrasound applicator during treatment, according to some embodiments of the invention;

FIG. 12E is a schematic illustration of a tissue holder ultrasound applicator with two separate cooling systems, according to some embodiments of the invention;

FIG. 12F is a schematic illustration of an angular tissue holder ultrasound applicator, according to some embodiments of the invention;

FIGS. 13A-13F are schematic illustrations of expanding ultrasound applicators, according to some embodiments of the invention;

FIGS. 14A-14B are schematic illustrations of a cooling system of an ultrasound applicator, according to some embodiments of the invention;

FIG. 15 is a schematic illustration of an ultrasound applicator with a plurality of TECs, according to some embodiments of the invention;

FIGS. 16A-16C are schematic illustrations of ultrasound transducers distribution on an outer surface of an ultrasound applicator, according to some embodiments of the invention;

FIGS. 17A-17B are schematic illustrations of ultrasound transducers patterns on an outer surface of an ultrasound applicator, according to some embodiments of the invention;

FIG. 18 is a schematic illustration of an ultrasound applicator with internal ultrasound transducers, according to some embodiments of the invention;

FIG. 19A is a schematic illustration of an ultrasound applicator with external mounter ultrasound transducers, according to some embodiments of the invention;

FIG. 19B is a schematic cross-section of the ultrasound applicator shown in FIG. 19A, according to some embodiments of the invention;

FIGS. 19C-19E are schematic illustrations of an ultrasound applicator, according to some exemplary embodiments of the invention;

FIG. 19F is a flow chart of a treatment procedure, according to some exemplary embodiments of the invention;

FIG. 20A is a schematic illustration showing the effect of applied ultrasonic energy on different layers of the skin while cooling the epithelium layers, according to some exemplary embodiments of the invention;

FIG. 20B are photos of an applicator used in an experiment and according to some exemplary embodiments of the invention;

FIG. 20C is a photo showing a treatment region within a vagina of a pig during an experiment;

FIG. 20D is a schematic illustration showing changes in the rotation orientation of the applicator within the vagina in an experiment, and according to some exemplary embodiments of the invention;

FIG. 20E is a group of photos and a schematic illustration showing burn marks on a skin of a pig performed by activation of ultrasound transducers during an experiment;

FIGS. 21A and 21B are photos of a vagina tissue from a sacrificed pig animal following experimental treatment, showing treated regions on the skin (FIG. 21B);

FIG. 21C is a group of an upper photo showing burn marks on the skin and a lower histochemical analysis image showing the damage to the epithelium and deep tissue layers;

FIG. 21D is a group of a photo showing a treated region on the skin and a lower histochemical analysis image showing damage to deep tissue regions without damaging the skin or epithelium layer;

FIGS. 22A and 22B are photos of an ultrasound applicator used in an experiment;

FIGS. 22C-22F are simulation results of the effect of ultrasonic energy application having different intensities and time durations on tissue; and

FIGS. 23A-23F are histochemical analysis images showing the effect of ultrasonic energy applied with different intensities and time durations on different tissue layers of the skin.

### DESCRIPTION OF SPECIFIC EMBODIMENTS OF THE INVENTION

The present invention, in some embodiments thereof, relates to female urogenital system treatments and, more particularly, but not exclusively, to tissue remodeling treatments in the urogenital system and/or anal incontinence treatments.

An aspect of some embodiments relates to affecting tissues of the female urogenital system by ultrasound energy. In some embodiments, the ultrasound energy, for example unfocused ultrasound energy, for example generated by unfocused ultrasonic waves, affects deep tissue layers. As used herein the term unfocused means non-converging. In some embodiments, unfocused ultrasonic waves are ultrasonic waves which are not focused to converge in two dimensions. On some embodiments, the ultrasound energy affects one or more tissue volumes in the tissue layers. In some embodiments, the ultrasound energy penetrates into depths equal or larger than 100 μm, for example 100 μm, 150 μm, 200 μm, 250 μm or any intermediate, smaller or larger depth. In some embodiments, the ultrasound energy is delivered into the vagina wall and/or into the Labia, for example into the major Labia or into the minor Labia. In some embodiments, the ultrasound energy heats deep tissue layers of the vagina, for example the Lamina propria tissue layer and/or the fibromuscular layer. Optionally, during the ultrasound energy treatment, superficial tissues, for example the Epithelium, are not affected by the thermal effect of the ultrasound energy. In some embodiments, the tissue contacting the ultrasound applicator is cooled. In some embodiments, the cooling is controlled and adjusted, for example according to the desired target depth in the tissue.

The Lamina propria is a layer of loose or dense connective tissue, and includes collagen which is important for compressibility and elasticity. The layer is rich in cells and provides support and nutrition to the epithelium layer, which is a more superficial layer. In addition, the Lamina propria layer is rich in blood cells and lymphatic channels and optionally includes afferent and efferent nerve endings.

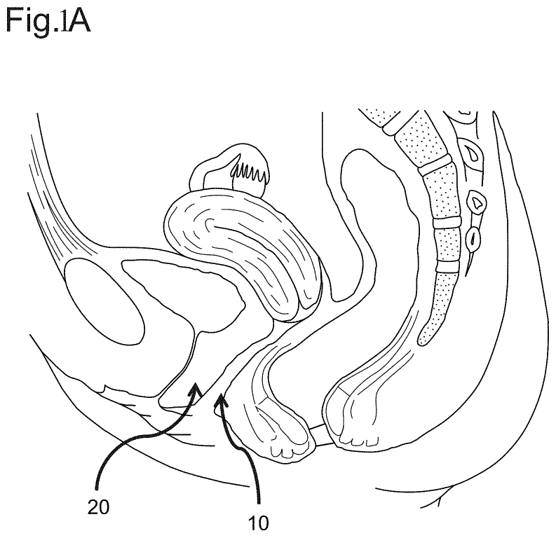

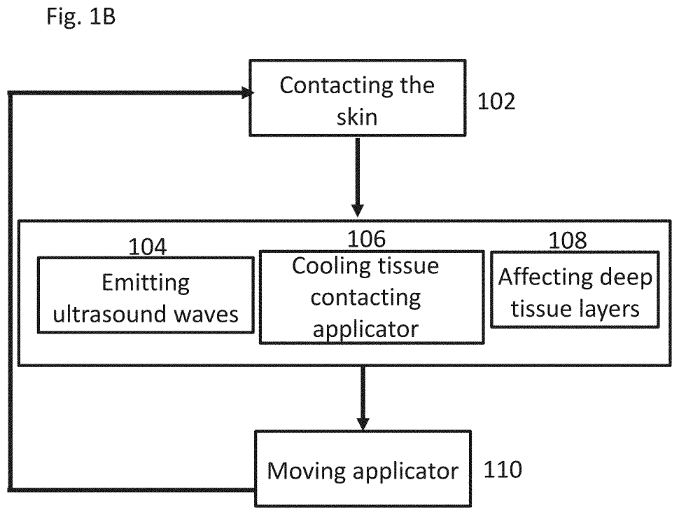

According to some embodiments, ultrasonic waves, are emitted from within the vagina. In some embodiments, for example as shown in FIG. 1A, emitting ultrasound waves from within the vagina 10 allows to treat target regions within the vagina wall, for example for vagina rejuvenation. Alternatively, or additionally, the emitted ultrasound waves treat target regions within the urethra 20 wall. Optionally, the emitted ultrasound waves treat target regions near the urethra wall 20, for example target regions located at a distance of up to 15 mm from the urethra wall. In some embodiments, ultrasonic waves with frequency lower than 11 MHz, for example lower than 10 MHZ, lower than 9 MHz or any intermediate, smaller or larger values are delivered to a tissue volume near the urethra.

According to some embodiments, the ultrasonic waves are emitted from transducers of an ultrasound applicator that is shaped and sized to be introduced at least partly into the vagina. In some embodiments, ultrasound transducers positioned close to the surface of the applicator and optionally near a distal end of the applicator emit the ultrasonic waves. In some embodiments, the ultrasound transducers emit the ultrasonic waves while placed in contact, optionally through a cover, with a treated tissue, for example with the vagina wall.

According to some embodiments, the ultrasonic waves are emitted towards one or more tissue volumes in the tissue. According to some embodiments, at least some of the ultrasonic waves cross each other. In some embodiments, the crossing point is located in the selected tissue volume. In some embodiments, the ultrasonic waves are emitted with a shift of at least 1° from a line of sight. Optionally, the ultrasonic waves are emitted with a shift of at least 1° from a line of sight relative to other ultrasonic waves.

According to some embodiments, at least some of the ultrasonic waves contact the selected tissue volume or different tissue volumes at spaced apart contact points positioned at a distance of up to 40 cm from each other, for example up to 10 cm, up to 5 cm, up to 2 cm, up to 0.1 cm or any intermediate, smaller or larger value, in the selected tissue volume or in spaced apart tissue volumes.

According to some embodiments, at least some of the one or more tissue volumes has a maximal dimension value, for example maximal length, maximal width, maximal diameter, maximal thickness of up to 30 cm, for example up to 20 cm, up to 10 cm, up to 1 cm, up to 0.1 cm or any intermediate smaller or larger value.

According to some embodiments, during the delivery of ultrasound energy into deep tissue layers, a superficial tissue layer, for example the epithelium is cooled. In some embodiments, the superficial tissue layer is cooled, for example to prevent heat damage or formation of thermal lesions on the superficial tissue layers. In some embodiments, the epithelium is cooled or frozen, for example to cause necrosis of the tissue. In some embodiments, the superficial tissue layers are cooled by cooling one or more ultrasound transducers contacting the tissue. Alternatively, or additionally, the superficial tissues are cooled by cooling at least some of the external surface of an ultrasound applicator contacting the superficial tissue. Optionally, the superficial tissue layers are cooled down by the ultrasound applicator through a cover layer placed between the applicator and the tissue.

According to some embodiments, the emitted ultrasonic waves are used to treat symptoms related to vulvo-vaginal tissue. In some embodiments, the symptoms occur following pathological conditions, for example cancer and/or or aging. Without being bound by any theory, with menopause, the ovaries stop producing the hormones estrogen and progesterone. As hormone levels drop, folds of the vagina lining become thinner, dryer less elastic, and possibly irritated with and/or without petechia. Optionally, the lining of the urethra also becomes thinner and may additionally lead to bladder control problems and urination disorders.

According to some embodiments, the emitted ultrasonic waves are used for treating one or more symptoms related to vaginal atrophy, for example thinning, drying and inflammation of the vaginal walls. Vaginal atrophy results when there is less estrogens present in genital tissues of post-menopausal women. The increase in vaginal atrophy due to lower circulating estrogens contributes to vaginal dryness, loss of pelvic support with resulting prolapse, decreased tissue elasticity and urogenital discomfort. In some embodiments, the emitted ultrasonic waves are used to treat stress urinary incontinence, for example by affecting tissue layers in the urethra wall or tissue layers near the urethra wall, for example tissue layers at a distance of up to 15 mm from the urethra wall.

According to some embodiments, when treating urinary incontinence by affecting tissue layers near or at the urinary wall, vaginal wall tissue contacting the ultrasound applicator is cooled. Alternatively or additionally, a catheter is introduced into the urethra and is configured to cool the urethra wall during the delivery of the ultrasonic waves from within the vagina. Alternatively or additionally, the catheter is configured to be used as a reflector for directing the ultrasonic waves from the vagina to a selected tissue volume. According to some embodiments, the reflected ultrasonic waves from the catheter are received by the ultrasonic transducers of the applicator. Measuring the intensity of the reflected waves can help direct the ultrasonic transducer towards the required location in the urethra, by rotating the applicator to get a maximal reading of the received reflection. Optionally or additionally, the distance between the ultrasonic transducer and the urethra can be measured by measuring the flight time of the ultrasonic wave to and from the reflecting catheter. This allows to accurately change the emitted ultrasonic treatment parameters, to reach the required ultrasonic intensity in the urethra, and thus to generate the required tissue heating. Alternatively, or additionally, the catheter is configured to measure the heating of the urethra wall temperature, and thereby to allow feedback of the tissue temperature near the urethra wall, and thereby to control the transducer ultrasonic emission to get the required tissue heating and effect. Alternatively, or additionally, the catheter is configured to measure the ultrasonic waves intensity in the urethra, to allow controlled treatment.

According to some embodiments, the emitted ultrasonic waves are used for treating one or more symptoms related to vaginal prolapse. In some embodiments, treating vaginal prolapse comprises one or more of tightening the tissue, strengthening the vaginal wall and/or increasing the blood flow to the area.

According to some embodiments, the emitted ultrasonic waves penetrate into the Lamina propria, and optionally lead to thermally induced collagen coagulation, and/or denaturation. Optionally, coagulation and/or denaturation of collagen results with contraction within lesions. In some embodiments, the creation of the lesions in thermal coagulation points (TCP) leads, optionally, to an inflammatory response, for example to an inflammatory wound-healing response. Optionally, the inflammatory response generates long-term tissue remodeling. In some embodiments, the long-term tissue remodeling leads to tightening of the Vagina wall.

According to some embodiments, heating of the Lamina Propria, which is part of the mucosa layer leads to contraction of collagen, collagen remodeling and/or increases elasticity. In some embodiments, the heating leads to long-term production of new collagen. Alternatively, the heating increases blood flow and/or restores nerve signaling. Optionally, restoration of nerve signaling results in normal vaginal lubrication.

An aspect of some embodiments relates to delivery of ultrasonic energy by a plurality of ultrasound transducers positioned on a non-planar surface, for example a curved surface of an ultrasound applicator. In some embodiments, the ultrasound transducers are radially spaced-apart on the non-planar surface. Additionally or alternatively, the ultrasound transducers are axially spaced-apart on the non-planar surface of the applicator. Optionally, the non-planar surface of the ultrasound applicator has a radius of curvature smaller than 5 cm, for example 3 cm, 2.5 cm, 2 cm, 1 cm or any intermediate, smaller or larger value.

According to some exemplary embodiments, mounting a plurality of transducers on a non-planar surface, allows, for example to emit ultrasonic waves at different circumferential range of angles larger than 90 degrees, for example 100 degrees, 110 degrees 180 degrees or any intermediate, smaller or larger values.

According to some embodiments, the plurality of transducers is mounted on one or more non-planar transducer bases. In some embodiments, the transducers base is a heat-conducting base, optionally made from a heat-conducting material for example aluminum. In some embodiments, the base is used as a heat-conducting interface between the transducers and at least one cooling element, for example a TEC. A potential advantage of using a heat-conducting base for a plurality of transducers, is that it allows to cool a plurality of transducers using a small number of TECs, and therefore optionally, to reduce the overall size of the transducer applicator.

An aspect of some embodiments relates to reshaping a tissue using ultrasound energy and application of force on the tissue. In some embodiments, the labia is reshaped by applying force and directing ultrasonic waves into the labia. In some embodiments, force is applied on one side or both sides of the Labia. In some embodiments, the force is applied on the Labia by at least one surface of an ultrasound applicator. Alternatively, the force is applied on the Labia by two opposite surfaces of an ultrasound applicator holding the Labia while delivering ultrasonic waves into the tissue.

An aspect of some embodiments relates to delivering ultrasonic energy into a wall of the vagina by emitting ultrasonic waves in circumferential range of angular directions larger than 90 degrees from within the vagina. In some embodiments, the ultrasonic waves are emitted from ultrasonic transducers arranged on an outer non-planar surface of an ultrasound applicator. In some embodiments, the ultrasound transducers are radially spaced apart on the outer non-planar surface of the ultrasound applicator. Additionally, the ultrasound transducers are axially spaced apart on the outer non-planar surface of the ultrasound applicator.

An aspect of some embodiments relates to selectively treating different anatomical regions within the vagina by adjusting the emission of ultrasound waves in different directions. In some embodiments, the different regions are treated simultaneously by the same ultrasound applicator. In some embodiments, the emission of ultrasound waves from the applicator is adjusted according to anatomical regions surrounding the applicator, for example to avoid an undesired thermal effect in specific tissue regions.

According to some embodiments, at least one parameter of the ultrasound waves is adjusted when simultaneously treating two or more anatomical regions. In some embodiments, a frequency of the ultrasonic waves is adjusted. Alternatively or additionally, the intensity of the ultrasonic waves is adjusted. In some embodiments, the angular direction in which the ultrasonic waves are emitted is adjusted, for example by deactivating one or more ultrasound transducers facing undesired directions.

According to some embodiments, the delivered ultrasonic energy induces an inflammatory response, for example when transmitting ultrasonic waves with low energy and for a short duration. Optionally, in order to induce the inflammatory response, the ultrasonic waves are delivered with intensity and/or frequency levels that generates mild heating of the tissue.

According to some embodiments, the tissue remodeling treatments in the urogenital system are delivered, for example, to women after birth and to post-menopausal women, and comprise one or more vaginal (canal) tightening, Labia rejuvenation, Stress Urinary Incontinence (SUI) treatment, Post-menopausal atrophy treatment and treating post radiation dryness.

According to some embodiments, in vaginal tightening treatments, the applied ultrasonic energy is used to treat vaginal looseness, that optionally affects sex life. In some embodiments, women treated for vaginal tightening are post partum women, that suffer, for example, from sexual gratification.

According to some embodiments, in Labia rejuvenation treatments, the applied ultrasonic energy is used to treat dissatisfaction with dissatisfaction with vulva appearance (sagging skin, pigmentation, texture). In some embodiments, women treated for labia rejuvenation are women in all ages having an increased portion of sagging skin increase with age, that suffer from sexual gratification, and decline in aesthetic, comfort and/or quality of life.

According to some embodiments, in SUI treatment, the applied ultrasonic energy is used to treat urine leakage during stress, for example in up to 50% of post menopausal women. In some embodiments, women treated for SUI suffer from a decline in one or more of comfort, quality of life, and/or sexual gratification.

According to some embodiments, in a post radiation dryness treatment, the applied ultrasonic energy is used to treat excessive dryness, for example in breast cancer survivors, suffering from discomfort and/or a decline in quality of life.

According to some embodiments, the delivered ultrasonic energy is used to treat symptoms related to moderate to severe genitourinary syndrome of menopause (GSM), comprising: vaginal dryness, vaginal burning, vaginal discharge, genital itching, burning with urination, urgency with urination, an increase in the number of urinary tract infections, urinary incontinence, light bleeding after intercourse, discomfort with intercourse, decreased vaginal lubrication during sexual activity, and/or shortening and/or tightening of the vaginal canal.

Potential advantages of using the method, system and ultrasound applicator describe herein may include, large area coverage in short time, which optionally allows short treatment times of up to 6 minutes, for example 3 minutes, 4 minutes, 5 minutes or any intermediate, smaller or larger time period for a full treatment. Additional potential advantages may include reduce burns and pain due to cooling of the epithelium, and smart and simple solution for sterility for example using off the shelf condoms.

According to some exemplary embodiments, the treatment described herein is a cosmetic treatment. In some embodiments, the ultrasound applicator is used when there is no functional problem and/or functional limitation in the subject receiving the treatment.

According to some exemplary embodiments, ultrasonic waves with intensity of 40-60 w/cm\hat{ }2, for example 40-45w/cm\hat{ }2, 45-55 w/cm\hat{ }2 or any intermediate, smaller or larger range of values are emitted for 1-3 seconds to penetrate into a sub mucosa layer within the vagina wall. In some embodiments, ultrasonic waves with intensity of 35-55 W/cm\hat{ }2 or any intermediate, smaller or larger range of values are emitted for 3-7 seconds to penetrate into adventitia layer within said vagina wall

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary Process for Affecting Female Urogenital System Tissue

According to some exemplary embodiments, an energy emitting device, for example an ultrasound applicator is introduced at least partly into the vagina of a female subject. In some embodiments, the ultrasound applicator is introduced into the vagina when providing a vagina rejuvenation treatment. In some embodiments, the ultrasound applicator is introduced into the vagina when treating one or more of genitourinary syndrome of menopause (GSM), stress urinary incontinence. Alternatively or additionally, the ultrasound applicator is introduced into the vagina for vaginal tightening vaginal birth or for vaginal tightening in non-menopausal women, for example for improving life quality. In some embodiments, during the treatment, ultrasound waves are emitted simultaneously at different directions, for example to affect target regions in the vagina wall. In some embodiments, the affected target regions are located deep within the vagina wall. In some embodiments, the emitted ultrasound waves do not affect superficial tissue layers, for example epithelium layers, that are optionally placed in contact with the applicator surface. Reference is now made to FIG. 1B depicting a process for affecting female urogenital system tissue, according to some exemplary embodiments of the invention.

According to some exemplary embodiments, an ultrasound applicator introduced at least partly into the vagina is placed in contact with a surface of the vagina at 102. Optionally, a cover, for example a sheath covering the applicator is placed in direct contact with the surface of the vagina. In some embodiments, the applicator is pushed against the surface of the vagina at a selected location within the vagina. In some embodiments, the selected location is positioned in a proximity to a selected target region within the vagina wall.

According to some exemplary embodiments, ultrasound waves are emitted from a plurality of transducers of the applicator at 104. In some embodiments, the ultrasound waves are emitted simultaneously at different directions, for example sideways, upper and/or lower directions. In some embodiments, the ultrasound waves are emitted at an angle in a range of 180-360 degrees, for example 190 degrees, 200 degrees, 360 degrees or any intermediate, narrower or wider angle.

According to some exemplary embodiments, a tissue region contacting the applicator or the cover of the applicator is cooled at 106. In some embodiments, a tissue, for example epithelium tissue of the vagina positioned in proximity or in contact with the applicator is cooled. In some embodiments, the epithelium contacting the applicator and/or the cover of the applicator is actively cooled by the applicator, for example to prevent thermal tissue damage. In some embodiments, the epithelium is cooled while the ultrasound waves are emitted. In some embodiments, a tissue region contacting the applicator or the cover of the applicator is cooled prior to and/or during and/or after the delivery of ultrasonic energy into the tissue. Optionally, the level of cooling is higher during the delivery of ultrasonic energy. In some embodiments, the tissue is cooled prior to the delivery of ultrasonic energy, for example to reduce blood flow to the treated region. Optionally, cooling the tissue to reduce blood flow is combined with application of a vasoconstricting material, for example cream, on the cooled region or near the cooled region.

According to some exemplary embodiments, the tissue is cooled by the applicator up to a depth of about 1 cm.

According to some exemplary embodiments, deep tissue layers are affected by the emitted ultrasound waves at 104. In some embodiments, tissue layers positioned in a distance of at least 200 μm for the applicator surface, for example at distance of at least 250 μm, at a distance of at least 350 μm, or any intermediate, smaller or larger distance from the applicator surface, are affected. In some embodiments, the deep tissue layers affected by the emitted ultrasound waves comprise one or more of the Lamina propria tissue layer, the fibromuscular tissue layer, the adventitia and/or the urinary wall. Alternatively or additionally, the deep tissue layers affected by the emitted ultrasound waves include at least one target area for treating urinary incontinence.

According to some exemplary embodiments, the applicator is moved at 110. In some embodiments, the applicator is axially moved to a different location within the vagina, for example to affect a different selected region within the vagina wall. Alternatively or additionally, the applicator is rotated, for example to place a different transducer in proximity to a selected target region.

### Exemplary System

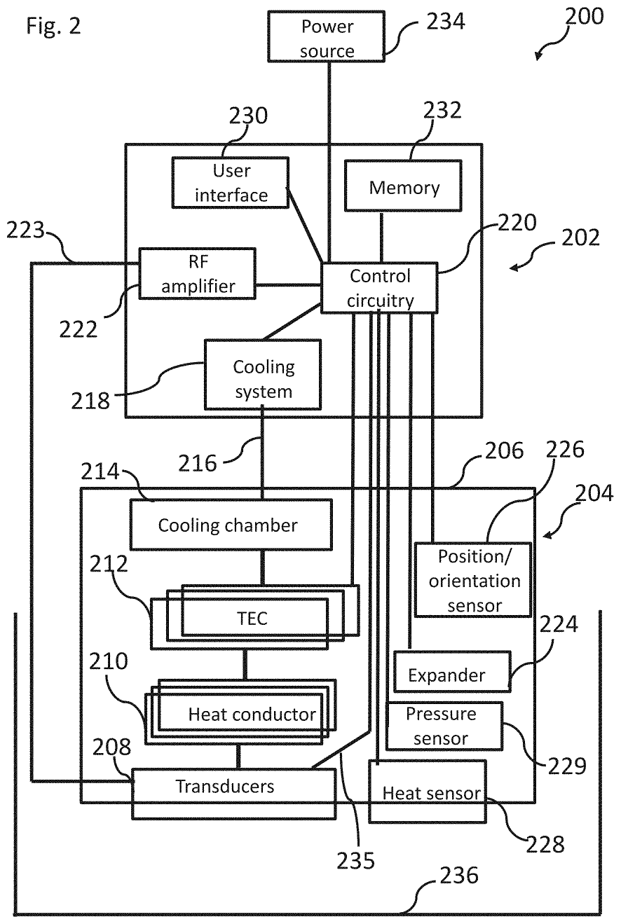

Reference is now made to FIG. 2, depicting a system for delivery of ultrasound energy to tissues of the female urogenital system, according to some exemplary embodiments of the invention.

According to some exemplary embodiments, the system 200 comprises an applicator 204 and a control unit 202, for example a portable control console. In some embodiments, the applicator 204 has a tubular body with a proximal end comprising a gripping member and a distal end, located away from the gripping member. In some embodiments, when introducing the applicator into a vagina, the distal end of the applicator is positioned within the vagina.

According to some exemplary embodiments, the tubular body of the applicator 204 is shaped and sized to be introduced at least partly into a female vagina. In some embodiments, the tubular body of the applicator has a maximal outer diameter or a maximal outer width in a range of 1-7 cm, for example 1-5 cm, 2-6 cm, 3-7 cm or any intermediate, smaller or larger range of values. In some embodiments, the tubular body of the applicator has a maximal length in a range of 4-20 cm, for example 3-12 cm, 5-15 cm, 7-20 cm or any intermediate, smaller or larger range of values. In some embodiments, the tubular body of the applicator is partly flexible, for example to allow bending of the applicator inside the vagina.

According to some exemplary embodiments, the applicator comprises one or more transducers 208, for example 4, 5, 10, 12 or any larger or smaller number of transducers. In some embodiments, the transducers 208 are ultrasound transducers, for example non-focused ultrasound transducers. In some embodiments, the transducers are thin transducers having a thickness of 0.1-2 mm, for example 0.1 mm, 0.5 mm, 1 mm, 1.5 mm or any intermediate, smaller or larger value. In some embodiments, the ultrasound transducers comprise piezoelectric ultrasound transducers. In some embodiments, the transducers are at least partly extending out from openings in a housing 206 of the applicator 204. In some embodiments, the transducers 208 are axially and/or radially distributed along the external surface of the applicator 204. Optionally, the transducers 208 are distributed along a surface of a distal region of the applicator located in a proximity to the distal end.

According to some exemplary embodiments, the transducers comprise one or more of a PZT, coating and/or at least one electrode.

According to some exemplary embodiments, one or more of the transducers generate ultrasonic waves and some of the transducers operate at a different frequency, for example to transfer most of the energy to heat energy. In some embodiments, transferring the energy emitted by the transducers to heat energy allows, for example, to avoid over cooling of the tissue in regions where ultrasound energy is not delivered. Alternatively, the transducers that do not deliver ultrasonic energy to the tissue are used to generate radio-frequency (RF), for example to heat untreated regions of the skin.

According to some exemplary embodiments, the applicator 204 comprises at least one heat conductor 210, configured to conduct heat away from the transducers 210. Alternatively or additionally, the heat conductor is configured to cool the housing 206 of the applicator 204. In some embodiments, the heat conductor 210 serves as a base layer for the transducers 208, and is shaped and sized to allow positioning of the transducers at a desired axial and/or radial orientation along the applicator. In some embodiments, the heat conductor 210 is made from a heat-conducting material, for example Aluminum, Brass or any other heat-conducting material that allows high levels of heat conduction.

According to some exemplary embodiments, the applicator 204 comprises at least one cooling module, for example a cooler. In some embodiments, the cooler comprises one or more thermoelectric coolers (TEC) 212. In some embodiments, the cooler or the TEC are configured to cool at least one ultrasound transducer of the applicator, and/or a tissue contacting the ultrasound transducer. In some embodiments, the TEC are attached to part of the at least one heat conductor 210. In some embodiments, the TEC 212 comprises a hot face and a cold face. In some embodiments, the cold face of the TEC is attached to the at least one heat conductor 210, for example to cool the heat conductor 210. In some embodiments, heat is conducted from a plurality of transducers 208 to a single TEC, for example TEC 212, through one or more heat conductors 210. Alternatively, a plurality of transducers 208 surround two or more TECs, and heat is conducted from the transducers through two or more heat conductors to the TECs.

According to some exemplary embodiments, the applicator 204 comprises at least one cooling chamber 214, which is part of a liquid-cooling system of the system 200. In some embodiments, the cooling chamber contacts at least partly the hot face of the TEC, for example to conduct heat from the hot face of the TEC to a coolant liquid within the cooling chamber 214. In some embodiments, the cooling chamber 214 is connected to a cooling system 218, optionally comprising a pump and a reservoir in the control unit 202 via tubing 216. In some embodiments, the pump circulates the coolant liquid between the reservoir in the control unit 202 and the cooling chamber 214 in the applicator 204. Optionally, the cooling system 218 comprises a chiller configured to dissipate heat from the circulating coolant liquid.

According to some exemplary embodiments, the control unit 202 comprises at least one amplifier 222 electrically connected to the transducers 208 by one or more electrical wiring 223, for example printed circuit board (PCBs) and/or flex PCBs. In some embodiments, the at least one Amplifier 222 is electrically connected to a control circuitry 220 of the control unit 202. Alternatively or additionally, the control circuitry 220 is electrically connected to the transducers by electrical wiring 235.

According to some exemplary embodiments, the control circuitry 220 controls the emission of ultrasonic waves from the transducers 235. In some embodiments, the control circuitry 220 controls the activation of the transducers, for example to activate transducers facing a first direction and not activating transducers facing a different direction. Alternatively or additionally, the control circuitry 220 controls the frequency of the ultrasonic waves. In some embodiments, controlling the frequency of the ultrasonic waves allows to emit low frequency ultrasonic waves at a first direction, for example to affect deep target regions, and emit high frequency ultrasonic waves at a second direction to affect more superficial target regions. In some embodiments, the control circuitry 220 controls the simultaneously emitting of ultrasonic waves with different parameters values at different directions. In some embodiments, the parameters comprise one or more of ultrasonic waves frequency, duration, rate of increase and ultrasonic waves direction.

According to some exemplary embodiments, the control circuitry 220 controls the activation of selected transducers, for example to reach a desired radial treatment band or a desired treatment band width. Alternatively or additionally, the control circuitry 220 controls the activation of selected transducers, for example to reach a desired treatment region size.

According to some exemplary embodiments, the control unit 202 comprises a memory 232, which stores at least one treatment protocol or parameters thereof. Alternatively or additionally, the memory stores values of at least one activation parameter of the transducers, for example frequency, intensity or duration. In some embodiments, the memory 232 stores indications related to anatomical structures and/or anatomical regions within the vagina. In some embodiments, the stored anatomical structures and/or anatomical regions are specific to a female subject, and are optionally obtained using one or more imaging analysis techniques, for example ultrasound imaging or magnetic resonance imaging (MRI).

According to some exemplary embodiments, the applicator 204 comprises at least one position and/or orientation sensor 226, for example an accelerometer. In some embodiments, the at least one position and/or orientation, for example rotation orientation, sensor is electrically connected to the control circuitry 220. In some embodiments, the at least one position and/or orientation sensor senses the position of the applicator 204 within the vagina. In some embodiments, the control circuitry 220 calculates an axial distance and/or a penetration depth of the applicator 204 within the vagina based on signals received from the at least one position and/or orientation sensor. Additionally or alternatively, the at least one position and/or orientation sensor 226 senses a rotation of the applicator 204 within the vagina. In some embodiments, the control circuitry 220 calculates a relative angle of the applicator 204 within the vagina. According to some exemplary embodiments, the control circuitry 220 is configured to calculate a penetration depth of said ultrasound applicator, or a body portion of the ultrasound applicator into the vagina, based on signals received from said at least one position and/or orientation sensor 226. Alternatively or additionally, the control circuitry 220 is configured to calculate a rotation angle of said ultrasound applicator or a body of said ultrasound applicator based on signals received from said at least one position and/or orientation sensor 226. In some embodiments, the control circuitry is configured to modify one or more parameter values of ultrasonic waves, based on said calculated penetration depth, position of said ultrasound applicator inside the vagina and/or said calculated rotation angle. In some embodiments, the one or more ultrasonic waves parameter comprise ultrasonic waves frequency, ultrasonic waves intensity, ultrasonic waves delivery time. In some embodiments, the control circuitry is configured to change cooling level of one or more of cooling module, one or more TECs, cooling level of tissue contacting the ultrasound applicator, based on said calculated penetration depth and/or said calculated rotation angle. Optionally, the control circuitry changes the cooling level and/or ultrasonic waves parameter values automatically.

According to some exemplary embodiments, the system 200 comprises at least one user interface, for example user interface 230 positioned in the control unit 202. In some embodiments, the user interface 230 is configured to deliver human detectable indications, for example audible and/or visual indications. In some embodiments, the user interface 230 comprises at least one display, for example for presenting information and/or delivery of visual indications to a user of the system 200. Additionally or alternatively, the user interface 230 comprises at least one speaker for example for delivery of alerts to a user of the system 200. In some embodiments, the user interface 230 is configured to receive input data from a user of the system 200, for example using the display or using a keyboard of the user interface 230.

According to some exemplary embodiments, the control circuitry 220 signals the user interface 230 to show on the display a calculated position and/or orientation of the applicator 204 within the vagina. In some embodiments, if a calculated position and/or a calculated orientation is not a desired position and/or a desired orientation, then the control circuitry 220 signals the user interface 230 to generate an alert signal.

According to some exemplary embodiments, the applicator 204 comprises at least one heat sensor, for example heat sensor 228 electrically connected to the control circuitry 220. In some embodiments, the at least one heat sensor 228, for example a thermistor, is configured to measure temperature of a tissue placed in contact with the applicator 204. In some embodiments, the at least one heat sensor 228 are thermally isolated from the applicator surface. In some embodiments, heat sensors of the applicator are spaced apart from each other, and are optionally thermally isolated from each other. Optionally, the heat sensor 228 measures the temperature of the tissue through an introducer, for example a cover, for example cover 236 covering at least partly the applicator 204.

According to some exemplary embodiments, the cover 236 has minimal thickness, and is optionally disposable. In some embodiments, the cover 236 allows for example minimal disturbance and/or absorbance of ultrasonic energy, for example during treatment. Additionally, the cover 236 allows minimal disturbance of heat transfer between the tissue and the face of the applicator 204 contacting the tissue. In some embodiments, the cover 236 is made from a hydrophilic material, for example to allow application of standard ultrasonic gel serving as a contact agent, on an outer surface of the cover. Alternatively, the cover 236 is made from hydrophobic material, for example to allow application of oil-based agents on the outer surface of the cover. In some embodiments, an oil based agent is applied on the cover. Alternatively, a water-containing gel is applied on the cover. In some embodiments, the introducer, for example the cover 236, is liquid sealed, for example to prevent contact between liquids within the vagina and a portion of the applicator positioned within the vagina.

According to some exemplary embodiments, a vaginal ultrasound applicator is covered with the cover 236, which is optionally a condom-like disposable cover, for example to maintain sterilization between different patients. In some embodiments, the ultrasound applicator will be covered with optionally 2 different layers of gel or liquid, for example to transfer the ultrasonic energy. In some embodiments, the two different layers are thin, for example to minimize the disturbance of contact cooling of the tissue and minimize possible disturbance and/or absorbance of ultrasonic energy. In some embodiments, air bubbles interfere with the delivery of ultrasonic energy and therefore need to be minimized. Optionally, the material used for the layers is with low viscosity.

According to some exemplary embodiments, the first layer is between the ultrasonic transducers face, and the cover 236. In some embodiments, the first layer is a layer of ultrasonic gel, optionally commercially available ultrasonic gel. Alternatively, the gel is made from paraffin oil, for example to minimize air bubbles.

According to some exemplary embodiments, the second layer is between the cover 236 and the vaginal epithelium. In some embodiments, the second layer is a thin layer of ultrasonic gel, optionally commercially available ultrasonic gel. Alternatively, the second layer is formed from special gels or liquids, optionally containing anti-infection agents, or other therapeutic materials. Additionally, the vagina epithelium is covered with vaginal discharge fluid.

According to some exemplary embodiments, if the temperature of a tissue contacting the applicator is higher than a predetermined value, the control circuitry 220 deactivates the transducers 208. Alternatively or additionally, the control circuitry 220 activates or increases the circulation of a coolant liquid between the applicator 204 and the cooling system 218, for example by controlling the activation of a pump, which is optionally an electric pump.

According to some exemplary embodiments, the applicator 204 comprises at least one expander 224, for example at least one spring or at least one balloon. In some embodiments, the expander is configured to expand the body of the, which is optionally elastic. Alternatively or additionally, the expander 224 is configured to push at least some of the transducer 208 against the vagina wall with force sufficient to ensure close contact between at least some of the transducers and the vagina wall. In some embodiments, the control circuitry 220 controls the expansion of the expander 224, optionally in response to signals received from the user by the user interface 230.

According to some exemplary embodiments, the expander, for example expander 224 expands at least a portion of the applicator body symmetrically, for example to allow contact of said entire portion with the vagina wall. Alternatively, the expander expands said applicator body asymmetrically, for example to allow contact of only a selected region of the body with the vagina wall. In some embodiments, the expander is configured to expand said applicator body to a volume larger in up to 60%, for example up to 20%, up to 40%, up to 50% or any intermediate, smaller or larger value from the original volume of the ultrasound transducer body.

According to some exemplary embodiments, the expander, for example expander 224 expands the applicator body, for example to apply force to stretch the vagina wall during the delivery ultrasonic energy.

According to some exemplary embodiments, the applicator 204 comprises at least one pressure sensor 229 electrically connected to the control circuitry 220. In some embodiments, the pressure sensor 229 is configured to sense the pressure applied by the applicator 204 on the vagina wall or on the Labia. In some embodiments, the control circuitry 220 calculates the pressure applied by the applicator 204 on the vagina wall or on the Labia. In some embodiments, if the pressure applied on the vagina wall or on the Labia is higher than a stored pre-determined value, then the control circuitry signals the user interface 230 to generate a human detectable indication, for example an alert signal.

According to some exemplary embodiments, the body of the applicator 204 comprises two movable portions pivotally connected by a hinge. In some embodiments, each of the two movable portion comprises one or more transducers 208. In some embodiments the transducers of one movable portion face transducers of the second movable portion. In some embodiments, the two movable portions are shaped and sized to clamp part of a tissue, for example part of the major and/or minor labia, between transducers of the two movable portions.

### Exemplary Differential Effect

According to some exemplary embodiments, ultrasonic waves, optionally non-focused ultrasonic waves penetrate into deep tissue layers. In some embodiments, while deep tissue layers are affected for the heat generated by the ultrasonic waves, superficial layers, for example layers that are placed in contact with the applicator or with ultrasound transducers of the applicator are not thermally affected.

According to some exemplary embodiments, the ultrasonic waves are emitted into the vagina wall which includes 4 tissue layers: (1) Stratified squamous epithelium lining, (2) Elastic lamina propria, (3) Fibromuscular layer, and (4) the Adventitia layer. The Stratified squamous epithelium lining makes at the vagina internal wall, and is 150-250 μm thick. The epithelium layer provides protection against mechanic friction, and is lubricated by cervical mucus since the vagina does not contain any glands. Estrogen stimulates the epithelial cells to secrete glycogen which is broken down into lactic acid as a defense mechanism against pathogens.

The Elastic lamina propria is a dense connective tissue layer which projects papillae into the overlying the epithelium, and contains small blood vessels (capillaries). The Elastic lamina propria layer thickness is 350-550 μm. The Elastic lamina propria and the epithelium form the mucosa layer.

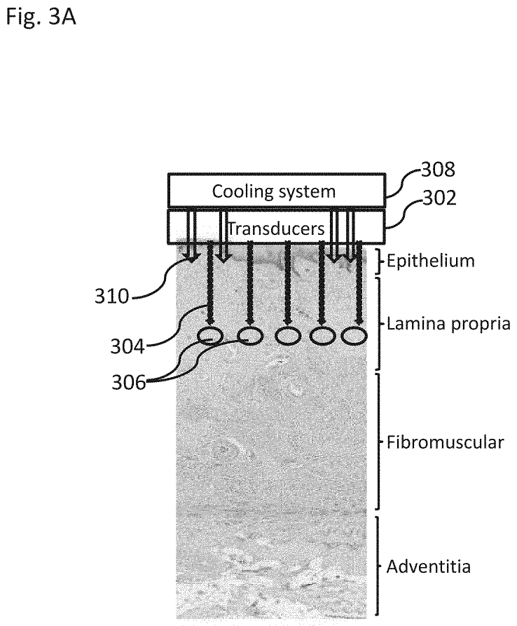

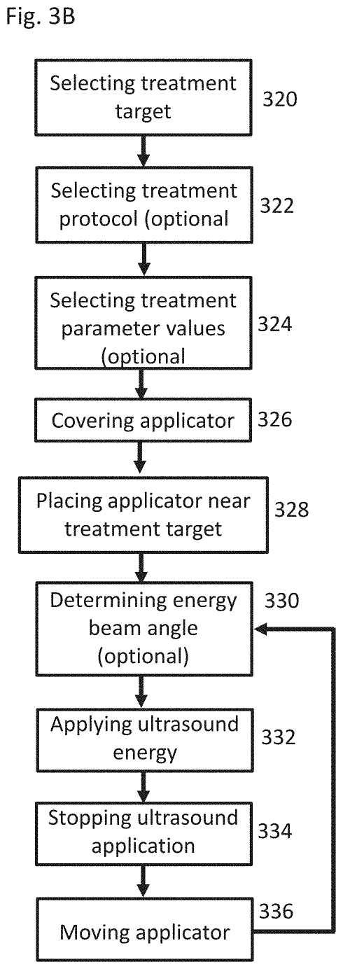

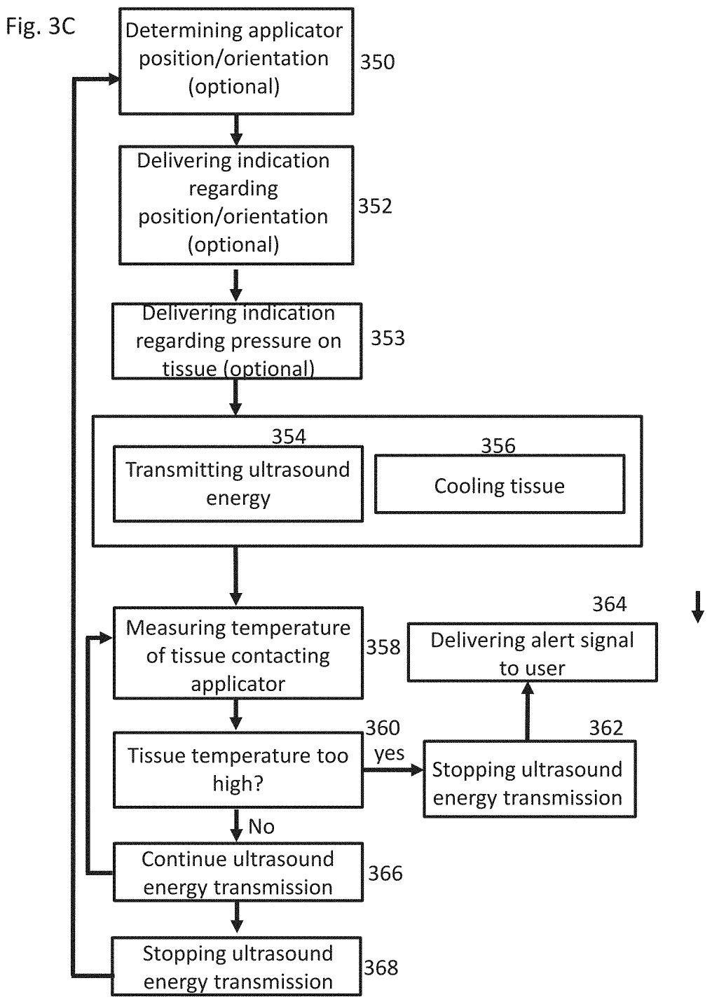

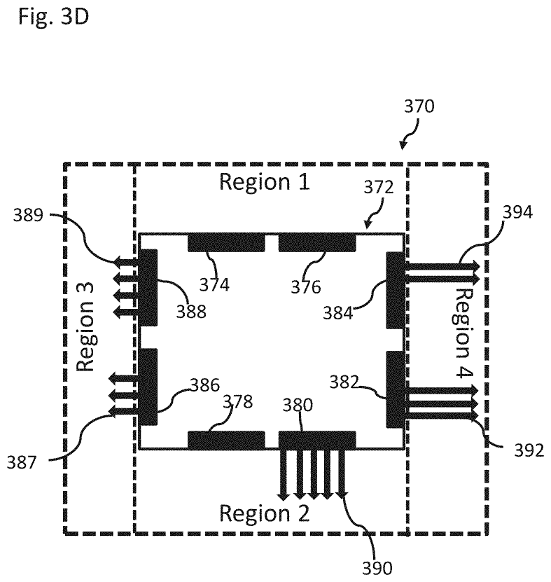

The Fibromuscular layer comprises two layers of smooth muscles. The Adventitia is a fibrous layer, which provides additional strength to the vagina wall, while also binding it to surrounding structures. Reference is now made to FIG. 3A depicting a selective differential effect, according to some exemplary embodiments.

According to some exemplary embodiments, transducers, for example ultrasound transducers 302 emit ultrasonic waves 304 with parameter values, for example frequency values that allow penetration of the ultrasonic waves 304 into the tissue. In some embodiments, the emitted ultrasonic waves 304 heat target regions 306 located at a distance of at least 200 μm from the transducers 302. Optionally, the target regions 306 thermally affected by the ultrasonic waves 304 are positioned at the Lamina propria tissue layer.

According to some exemplary embodiments, during the emitting of the ultrasonic waves, a cooling system 308 keeps the epithelium layer that is in a close proximity to the transducers 302 or in contact with the transducers 302 cool. In some embodiments, the cooling system keeps the external surface of the applicator which contacts the epithelium cool enough to prevent thermal damage of the epithelium. In some embodiments, the cooling system cools the external surface of the applicator to a temperature in a range between 5° C.-35° C., for example 5° C.-20° C., 10° C.-30° C., 15° C.-35° C. or any intermediate, smaller or larger range of values. Optionally, the temperature of the external surface of the applicator depends on the intensity and duration of the ultrasound emission. In some embodiments, cold waves 310 are conducted from the cooling system 308, through the external surface of the applicator into the epithelium tissue.

### Exemplary Detailed Usage Process

Reference is now made to FIG. 3B, depicting a detailed usage process, according to some exemplary embodiments of the invention.

According to some exemplary embodiments, a treatment target is selected at 320. In some embodiments, a user selects a treatment target within the vagina or at the minor/major labia. Alternatively, the user selects a treatment target in a tissue related to the female urogenital system.

According to some exemplary embodiments, a treatment protocol is optionally selected at 322. In some embodiments, the treatment protocol is selected based on the selected treatment target. In some embodiments, the treatment protocol is selected, for example to adjust the treatment to anatomical features at the selected treatment target and/or surrounding the selected treatment target, for example to avoid damage to nerve tissue. In some embodiments, the treatment protocol is stored in the memory 232 shown in FIG. 2.

According to some exemplary embodiments, treatment parameter values are selected at 324. In some embodiments, the treatment parameter values comprise intensity of ultrasound waves, frequency of ultrasound waves, direction of emitted ultrasonic waves and/or duration of a treatment session in which ultrasonic waves are emitted.

According to some exemplary embodiments, the applicator is covered at 326. In some embodiments, the applicator is covered by cover 236 shown in FIG. 2. In some embodiments, once the applicator is covered, a wetting material, for example a water-based lubrication material or oil-based material is applied on the outer surface of the cover in order to reduce friction between the cover and tissue surface.

According to some exemplary embodiments, the applicator is placed near treatment target at 328. In some embodiments, the applicator is introduced into the vagina, and is positioned in a close proximity to a selected treatment target. Additionally, once reaching a desired position within the vagina, the applicator is pushed against the vagina wall, for example to ensure good contact between the applicator and the vagina wall during treatment.

According to some exemplary embodiments, the energy beam angle is determined at 330. In some embodiments, the energy beam angle between some transducers of the applicator and the selected treatment region is selected, for example to reduce thermal damage in other tissue regions. In some embodiments, a user selects which transducers to activate and/or ultrasonic waves parameter values in the activated transducers, for example to control the thermal effect generated by the ultrasonic waves in different tissue regions.

According to some exemplary embodiments, ultrasound energy is applied at 332. In some embodiments, ultrasonic waves, for example unfocused ultrasonic waves are emitted at 332.

According to some exemplary embodiments, the ultrasound energy application is stopped at 334. In some embodiments, the ultrasound energy application is stopped after a pre-determined time period. Alternatively, the ultrasound energy application is stopped when a temperature level of tissue contacting the applicator is higher than a pre-determined temperature level.

According to some exemplary embodiments, the applicator is moved at 336, for example to reach additional target regions. In some embodiments, the applicator is axially moved within the vagina. Alternatively or additionally, the applicator is rotated within the vagina. In some embodiments, when reaching a new position on the tissue surface or within the vagina, the energy beam angle is optionally determined at 330.

### Exemplary Device Activation Process

Reference is now made to FIG. 3C, depicting a device activation process according to some exemplary embodiments of the invention.

According to some exemplary embodiments, after the insertion of the applicator into the vagina, the position and/or the orientation of the applicator is determined at 350. In some embodiments, the position and/or orientation of the applicator are determined by at least one sensor, for example sensor 226 shown in FIG. 2. Alternatively or additionally, the position and/or orientation of the applicator are determined using one or more markings on the external surface of the applicator. In some embodiments, the insertion depth of the applicator is determined at 350. Alternatively and/or additionally, the rotation angle of the applicator is determined at 350.

According to some exemplary embodiments, an indication related to the determined position and/or orientation is optionally delivered to a user at 352. In some embodiments, the indication, for example a human detectable indication is delivered optionally by the user interface 230 shown in FIG. 2.

According to some exemplary embodiments, an indication related to the pressure applied by the applicator on a tissue is optionally delivered to a user at 353. In some embodiments, if the force applied by the applicator on the tissue is higher than a pre-determined value, an alert signal is generated, for example by the user interface 230 shown in FIG. 2. Alternatively or additionally, if the force applied by the applicator on the tissue is lower than a pre-determined value, an indication is delivered to a user, for example to allow correction of the applicator position on the target skin area.

According to some exemplary embodiments, ultrasound energy is transmitted at 354. In some embodiments, the ultrasound energy is transmitted from a plurality of transducers of the applicator. In some embodiments, the ultrasound energy is transmitted from a plurality of transducers radially and/or axially distributed on the surface of the applicator.

According to some exemplary embodiments, the tissue contacting the applicator is cooled at 356 during the transmittance of ultrasound energy. Optionally, the tissue contacting the tissue is cooled prior to and during the transmittance of ultrasound energy. In some embodiments, the outer surface of the applicator is cooled by a cooling system, for example by at least one TEC and/or by a liquid cooling system within the applicator.

According to some exemplary embodiments, the temperature of the tissue contacting the applicator is measured at 358. In some embodiments, the tissue temperature is measured by at least one temperature sensor, for example a thermistor, optionally positioned at a contact point between the applicator and the tissue.

According to some exemplary embodiments, the system determines whether the tissue temperature is too high at 360. In some embodiments, a control circuitry of the system, for example control circuitry 220, shown in FIG. 2, compares measured temperature values to temperature values stored in a memory of the system. Optionally, the control circuitry compares the measured values to a look-up table stored in a memory of the system.

According to some exemplary embodiments, if the measured temperature values of the tissue are higher than a pre-determined value, the ultrasound energy emission is stopped at 362. In some embodiments, the ultrasound energy emission is stopped to prevent thermal damage to superficial tissue layers, for example the epithelium layer contacting the applicator. According to some exemplary embodiments, if the tissue temperature is lower than a certain temperature (for example OC, the cooling of the ultrasonic transducer by the TEC is decreased, in order to prevent damage to the tissue.

According to some exemplary embodiments, when the ultrasound energy emission is stopped, an alert signal is delivered to the user at 364.

According to some exemplary embodiments, if the measured temperature levels are lower than a predetermined value then the ultrasound energy emission continues at 366. In some embodiments, the ultrasound energy emission continues while constantly measuring the temperature of the tissue contacting the applicator.

According to some exemplary embodiments, the ultrasound energy transmission is stopped at 368. In some embodiments, the energy transmission is stopped, for example when reaching pre-determined treatment duration. In some embodiments, the treatment duration is determined based on the tissue type, the distance between the ultrasound transducers and the selected treatment target and/or the distance between the ultrasound transducers and anatomical regions surrounding the selected treatment target.

### Exemplary Differential Effect

According to some exemplary embodiments, the applicator comprises a plurality of ultrasound transducers radially distributed on the surface of the applicator. In some embodiments, when inserting the applicator into the vagina, the transducers face different anatomical regions. In some embodiments, the system adjusts the emission of the ultrasound waves according to the tissue type and/or anatomical region facing the transducers, for example to selectively treat different treatment regions. Reference is now made to FIG. 3D depicting a differential effect on tissue regions surrounding the applicator, according to some exemplary embodiments of the invention.

According to some exemplary embodiments, an applicator 372 comprises a plurality of transducers, for example transducers 374, 376, 378, 380, 382, 384, 386 and 388, facing at different directions. In some embodiments, when inserting the applicator 372 into a body lumen, for example into the vagina 370, the transducers face different anatomical regions, for example regions 1-4. In some embodiments, the anatomical regions facing the transducers are radially spaced apart from each other. Alternatively or additionally, the anatomical regions facing the transducers are axially spaced apart from each other. In some embodiments, parameter values of the ultrasonic waves emitted from each or some of the transducers are adjusted according to the anatomical region facing the transducers, for example to prevent undesired thermal damage in anatomical regions that include organs or tissue layers, for example nerve layers that should not be affected.

According to some exemplary embodiments, transducers 374 and 376 of the applicator 372 face anatomical region 1 within a wall of the vagina 370. In some embodiments, during the activation of other transducers of the applicator, the transducers facing anatomical region 1 are not activated, for example to prevent thermal damage to organs and tissue layers within region 1.

According to some exemplary embodiments, transducers 378 and 380 of the applicator 372 face anatomical region 2. In some embodiments, only part of the anatomical region 2 include areas that should not be thermally damaged. In some embodiments, only some transducers, for example transducer 380, are activated and emit ultrasonic waves 390, while others, for example transducer 378 are not activated.

According to some exemplary embodiments, in some anatomical regions, for example anatomical region 3, the selected treatment regions are positioned in superficial and less deep tissue layers. In some embodiments, transducers 386 and 388 facing anatomical region 3, emit ultrasonic waves 387 and 389 with parameter values that allow penetration into relatively superficial layers, and not into deep layers within the anatomical region 3.

According to some exemplary embodiments, in some anatomical regions, for example anatomical region 4, the selected treatment regions are positioned in deep tissue layers. In some embodiments, at least some of the transducers facing anatomical region 4, for example transducers 382 and 384 emit ultrasonic waves 392 and 394 with parameter values that allow penetration into deep tissue layers.

According to some exemplary embodiments, the activation of the transducers, for example transducers 374, 376, 378, 380, 382, 384, 386 is controlled by a control circuitry of the system, for example control circuitry 220 shown in FIG. 2. In some embodiments, the control circuitry activates the transducers according to parameter values stored in memory, for example memory 232. In some embodiments, the ultrasonic waves parameters comprise one or more of intensity, frequency, duration and/or rate of increase. In some embodiments, in order to treat relatively superficial targets, the ultrasonic waves have intensity levels in a range of 10-40 W/cm\hat{ }2, for example 10-20 W/cm\hat{ }2, 15-30 W/cm\hat{ }2 or any intermediate, smaller or larger values, and frequency levels in a range of 10-15 MHz, for example 10 MHz, 13 MHz, 15 MHz or any intermediate, smaller or larger value. In some embodiments, in order to treat deep tissue layers, the ultrasonic waves have intensity levels in a range of 1-10 MHz, for example 1 MHz, 5 MHz, 7 MHz, 10 MHz or any intermediate, smaller or larger value, and intensity levels in a range of 10-50 W/cm\hat{ }2, for example 10-20 W/cm\hat{ }2, 15-30 W/cm\hat{ }2, 20-50 W/cm\hat{ }2, or any intermediate, smaller or larger value.

Exemplary Applicator with Radially Displaced Transducers

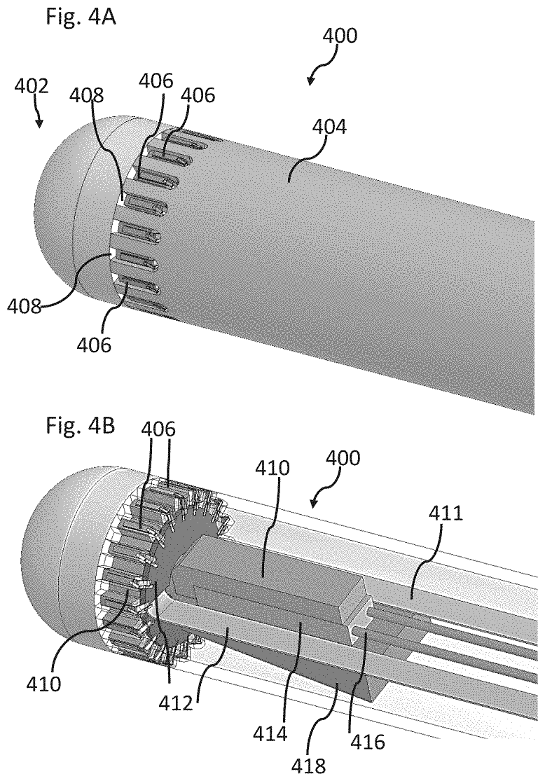

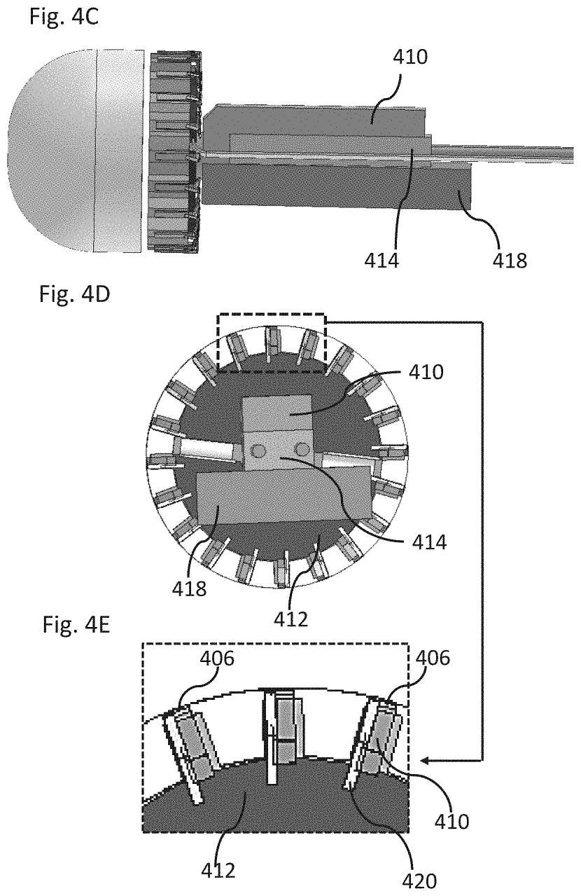

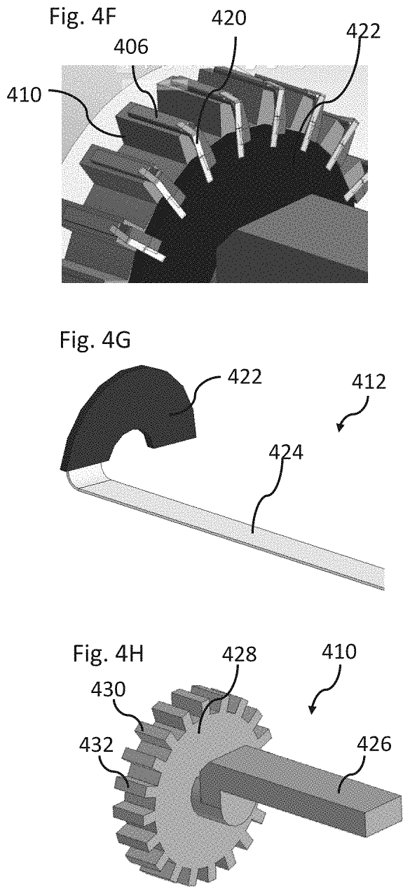

According to some exemplary embodiments, an ultrasound applicator comprising a plurality of ultrasound transducers, is shaped and sized to be introduced into a vagina, for example to allow vagina rejuvenation treatments. In some embodiments, the ultrasound transducers are radially distributed on the surface of the ultrasound applicator, for example to allow delivery of ultrasonic waves at different angular directions, optionally simultaneously delivery of the ultrasonic waves. Reference is now made to FIGS. 4A-4H, depicting an ultrasound applicator comprising radially distributed ultrasound transducers, according to some exemplary embodiments of the invention.

According to some exemplary embodiments, for example as shown in FIG. 4A, an ultrasound applicator 400 comprises an elongated tubular body 404 with a distal end 402 and an internal lumen. In some embodiments, the elongated tubular body 404 has a non-planar outer surface, optionally with a curvature radius smaller than 3 cm, for example 2.5 cm, 2 cm, 1 cm or any intermediate, smaller or larger curvature radius.

According to some exemplary embodiments, the ultrasound applicator 400 comprises a plurality of ultrasound transducers, for example transducers 406 radially distributed on a circumference of said tubular body 404. In some embodiments, the transducers 406 are radially distributed along the entire circumference of the ultrasound applicator 400. In some embodiments, for example as shown in FIG. 4A, the transducers 406 have the same axial position but a different radial position, for example to allow emitting of ultrasonic waves at angular directions larger than 90 degrees, for example 100 degrees, 180 degrees, 360 degrees or any intermediate, smaller or larger angle.

According to some exemplary embodiments, the transducers 406 are positioned within openings 408 in the outer surface of the tubular body 404. Optionally, the radially distributed transducers are positioned near the distal end 402 of the tubular body 404.

According to some exemplary embodiments, the radially distributed transducers 406 are organized side by side. In some embodiments, the transducers 406 are spaced apart from each other. Alternatively, the transducers 406 are at least partly contacting each other.

According to some exemplary embodiments, each of the ultrasound transducers is positioned on at least one base, for example a heat-conducting base. In some embodiments, for example as shown in FIG. 4B, each the transducers 406 is positioned on a single base, for example base 410 which is optionally a heat conducting base. In some embodiments, the heat-conducting base is made from a heat-conducting material, for example Aluminum, Brass or any other heat-conducting material that allows high levels of heat conduction. According to some exemplary embodiments, the heat-conducting base 410 is attached to a cooling element, for example a thermoelectric cooler (TEC) 414. In some embodiments, the heat-conducting base 410 is attached to a cold face of the TEC 414, for example to allow cooling of the transducers 406 via the heat-conducting base 410. In some embodiments, the TEC is electrically connected to a control unit of the applicator, for example control unit 202 shown in FIG. 2.

According to some exemplary embodiments, the hot face of the TEC is attached to a chamber, for example cooling bath 418 of a coolant liquid, for example water. In some embodiments, water circulates between the cooling bath 418 in the lumen of the applicator 400 and an external cooling system, for example cooling system positioned in a control unit of the ultrasound applicator, for example control unit 202 shown in FIG. 2. In some embodiments, a pump, for example an electric water pump circulates the water between the applicator 400 and a control unit or an external cooling system via tubes connected to the cooling bath 418.

According to some exemplary embodiments, for example as shown in FIGS. 4D-4G, each of the transducers 406 is attached to the base 410 and is electrically connected via electrical wiring or electrical strips, for example electrical strips 420 to a printed circuit board (PCB) or a Flex-PCB, for example PCB 412. In some embodiments, the applicator 400 comprises a single PCB or two or more PCBs. In some embodiments, for example as shown in FIG. 4G, the PCB 412 comprises a circular portion 422 configured to electrically connect the transducers, and an axial portion 424 configured to electrically connect the circular portion with a control unit of the applicator 400, for example control unit 202 shown in FIG. 2.

According to some exemplary embodiments, for example as shown in FIG. 4H, the base 410 comprises an axial base portion 426, having a flat surface shaped sized to fit a surface of a cooling element, for example the TEC 414, optionally to allow efficient heat and/or cold transfer between the base 410 and the TEC 414. In some embodiments, the base 410 comprises a round portion 428 with a plurality of protrusions, for example protrusions 430 and 432. In some embodiments, the protrusions which optionally have an upper flat surface serve as a base or a holder for the ultrasound transducer. In some embodiments, each of the transducers, which optionally have at least one flat surface and are optionally thin, is attached to the upper flat surface of the base protrusions. In some embodiments, having flat surface connections between heat-conducting elements, for example between a surface of the transducers and a surface of the base protrusions, allows maximal contact area to achieve efficient heat transfer between the contacting elements.

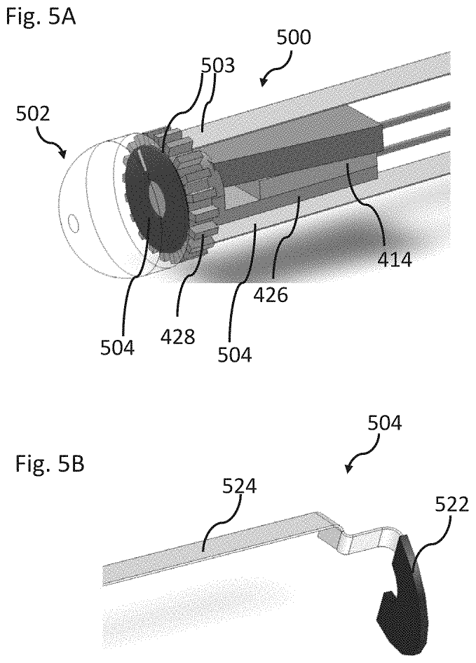

According to some exemplary embodiments, for example as shown in FIGS. 4D and 4F, the circular portion 422 of the PCB is attached to an inner side of the base 410, facing the TEC 414. In some embodiments, for example as shown in FIGS. 5A-5B, an ultrasound applicator 500 comprises two PCBs 503 and 504. In some embodiments, each of the PCBs, for example as shown in FIG. 5B in relation to PCB 504, comprises a circular portion 522 and an axial portion 524. In some embodiments, the circular portions of the PCBs 503 and 504, for example circular portion 522, are attached to the front surface of the round base portion 428 facing a distal end 502 of the applicator 500.

Exemplary Applicator with Radially and Axially Displaced Transducers

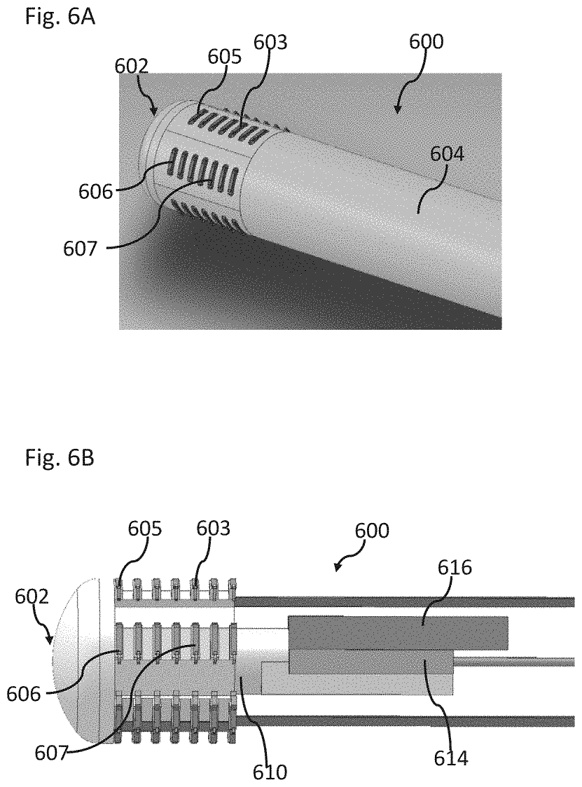

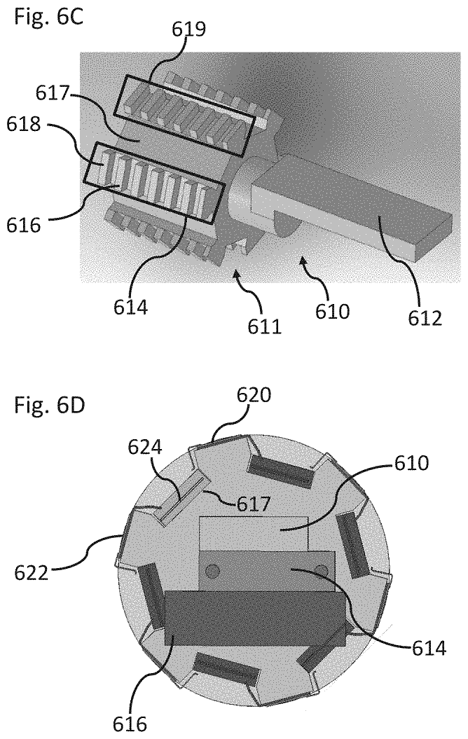

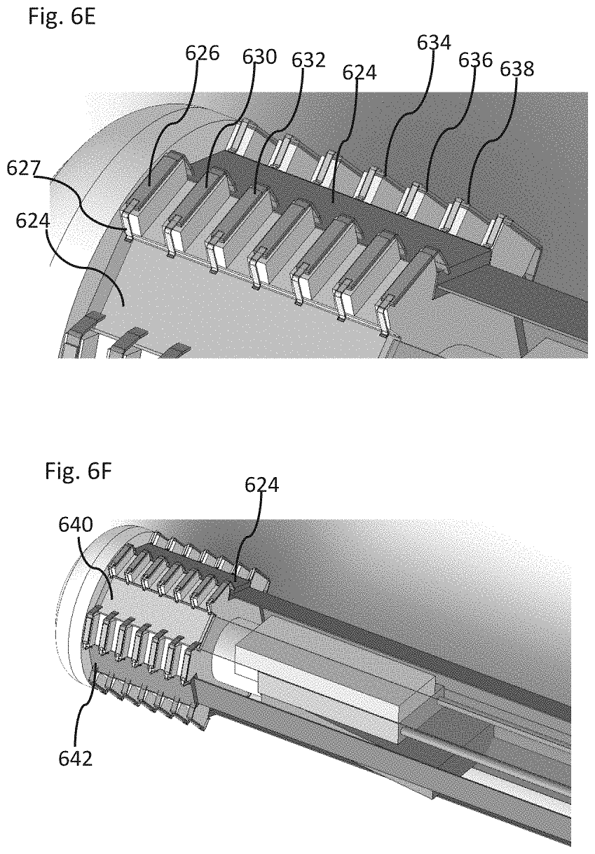

According to some exemplary embodiments, an ultrasound applicator comprises a plurality of ultrasound transducers distributed on the surface of the applicator. In some embodiments, some of the transducers are radially spaced apart from each other. Alternatively or additionally, some of the transducers are axially spaced-apart. In some embodiments, having both radially and axially spaced apart transducers allows, for example widening of a treatment strip. In some embodiments, widening the treatment strip allows, for example to treat a larger area with a fewer movements of the applicator in an axial direction. Reference is now made to FIGS. 6A-6F depicting an ultrasound applicator with radially and axially displaced transducers, according to some exemplary embodiments of the invention.

According to some exemplary embodiments, for example as shown in FIG. 6A, an ultrasound applicator, for example applicator 600 comprises an elongated body 604, optionally tubular or cylindrical body with a distal end 602. In some embodiments, the applicator 600 comprises a plurality of transducers arranged on the surface of applicator body 604. In some embodiments, some of the transducers have the same radial position but are axially spaced apart, for example transducers 606 and 607, or transducers 605 and 603. In some embodiments, some of the transducers have the same axial position but are radially spaced apart, for example transducers 605 and 606.

According to some exemplary embodiments, for example as shown in FIG. 6B, the transducers are attached to a base 610 which is optionally a heat-conducting base. In some embodiments, a surface of the base 610 is attached to a surface of a single TEC, for example TEC 614. In some embodiments, the base 610 is attached to a cold face of the TEC 614, for example to cool all the transducers that attached to the base, for example transducers 603, 605, 606 and 607. In some embodiments, a single TEC cools all the transducers of an applicator through a shared heat-conducting transducers base, for example base 610 also shown in FIG. 6C. In some embodiments, a hot surface of the TEC 614 is attached to a surface of a cooling bath 616 which includes coolant liquid, for example water, as discussed in FIGS. 4B-4D.

According to some exemplary embodiments, for example as shown in FIG. 6C, the base 610 of the applicator 600 comprises an axial portion, which is optional flat and is shaped and sized to fit a surface of a TEC. Additionally, the base 610 comprises a round portion 611 which includes groups, for example group 614, of axially spaced apart protrusions, for example protrusions 616 and 618. In some embodiments, each of the protrusions of the round portion 611 includes an upper surface, optionally a flat surface which is shaped and sized to allow mounting of an ultrasound transducer. In some embodiments, the round portion 611 of the base comprises several groups of axially spaced apart protrusions, for example groups 614 and 619. In some embodiments, a recess 617 is positioned between each two groups of protrusions.

According to some exemplary embodiments, for example as shown in FIG. 6D, the applicator comprises a single heat-conducting base, for example base 610 and a single TEC for cooling ultrasound transducers, for example transducers 620 and 622 attached to the protrusions of the base. In some embodiments, the transducers, for example radially spaced-apart transducers, are electrically connected by at least one flex-PCB placed in the recess 617 between each group of protrusions.

According to some exemplary embodiments, for example as shown in FIG. 6E, each transducer, for example transducer 626, is electrically connected to the PCB 624 by an electrical connector, for example an electric connector 627. In some embodiments, each PCB electrically connects radially spaced apart transducers for example transducers 626, 630, and 632 and transducers 634, 636 and 638. In some embodiments, each ultrasound applicator comprises a plurality of flex-PCBs, for example PCBs 624, 640 and 642.

According to some exemplary embodiments, an ultrasound applicator, for example ultrasound applicator 700 comprises an elongated body 704 and a plurality of ultrasound transducers radially distributed next to the body surface. In some embodiments, the plurality of transducers, for example are axially spaced apart, for example transducers 708 and 710 or transducers 712 and 714. Additionally, the plurality of transducers, for example transducers 708 and 712 are radially spaced-apart.

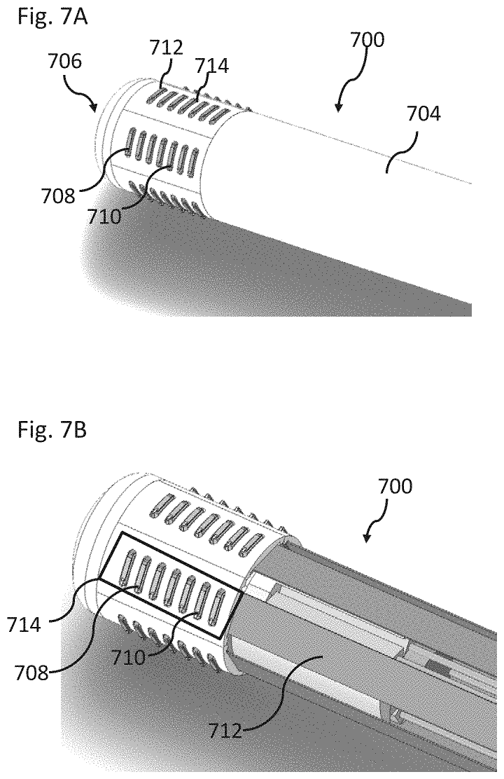

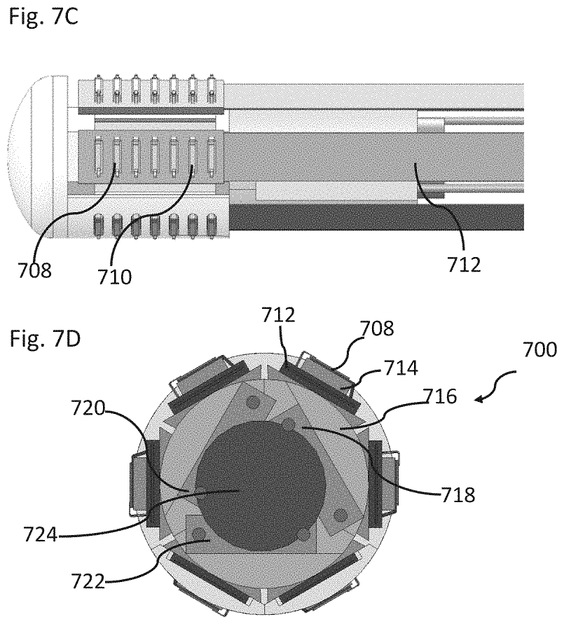

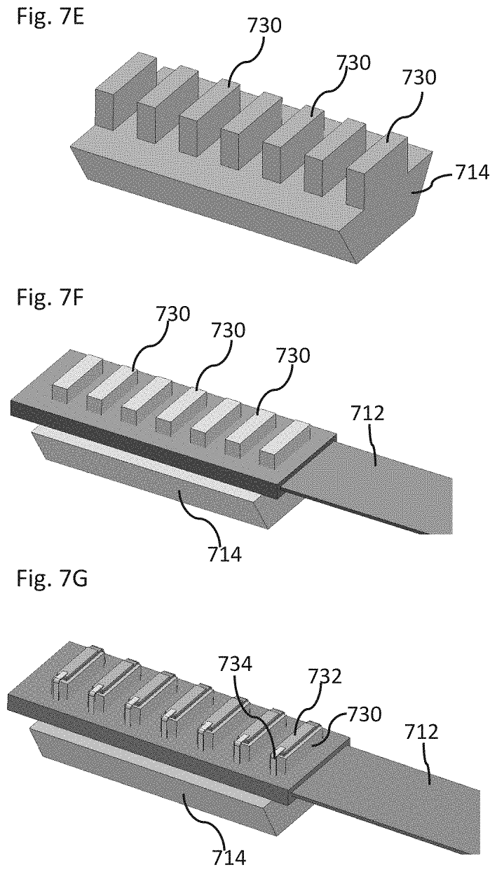

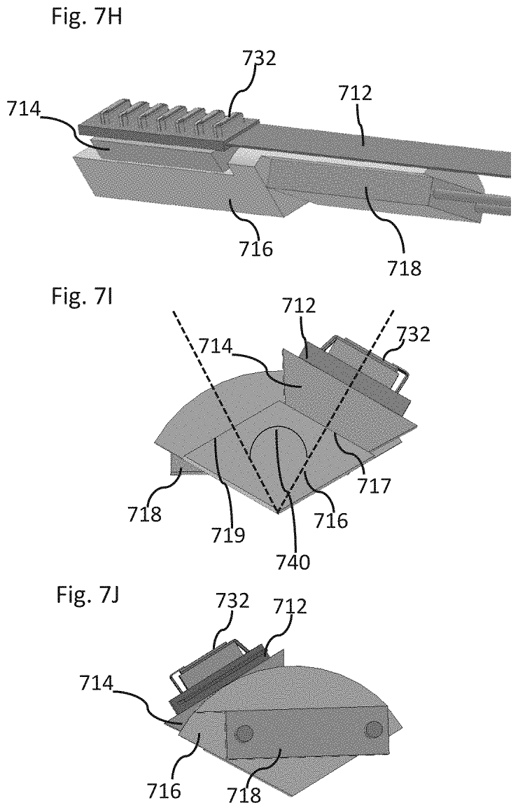

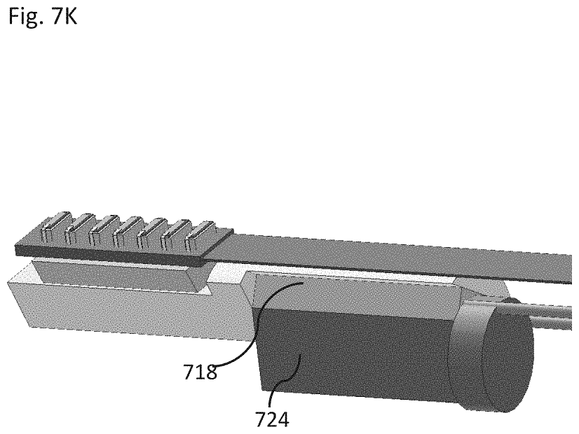

According to some exemplary embodiments, for example as shown in FIGS. 7B and 7C, transducers that are axially spaced apart but have the same radial position, for example transducers 708 and 710, optionally arranged as a group 714, are electrically connected to a single flex PCB, for example PCB 712. In some embodiments, electrically connecting a plurality of transducers to the same PCB, allows for example to activate the connected transducers together as a group, and optionally to increase the size of the treated area.

According to some exemplary embodiments, for example as shown in FIG. 7D, each transducer, for example transducer 708 is mounted on top of a base adaptor, for example base adaptor 714. In some embodiments, the base adaptor is a heat conducting base adaptor, optionally made from a heat-conducting material, for example aluminum. In some embodiments, a group of axially spaced apart transducers are mounted on the same base adaptor. In some embodiments, two of the base adaptors are connected through a base, for example base 716 to a single TEC, for example TEC 718. In some embodiments, for example as shown in FIG. 7D, the ultrasound applicator 700 comprises two or more TECs, for example 3 TECs 718, 720, and 722, In some embodiments, a hot surface or a hot face of the two or more TECs is attached to a single cooling bath, for example water bath 724.

According to some exemplary embodiments, for example as shown in FIGS. 7E-7G, a PCB, for example PCB 712 comprises openings that allow passage of the protrusion 730 of the base adaptor 714. In some embodiments, the PCB openings allow to attach the PCB to the upper surface of the base adaptor, in a close proximity to the protrusions 730. In some embodiments, for example as shown in FIG. 7G, ultrasound transducers, for example transducer 732 mounted on top of an upper flat surface of the protrusion 730, is electrically connected to the PCB by electrical connector 734.

According to some exemplary embodiments, two groups or axially spaced-apart transducers are connected through a shared heat conducting base to a single TEC. In some embodiments, the applicator comprises two or more TECs, for example to increase the efficiency of heat dissipation and/or to increase the efficiency of cooling the transducers by the TECs. In some embodiments, for example as shown in FIGS. 7H-7J, a single TEC, for example TEC 718 cools two groups of ultrasound conductors by a shared heat-conducting base, for example base 716.

According to some exemplary embodiments, the heat-conducting base 716 comprises two flat surfaces, for example surfaces 717 and 719, shaped and sized to allow the mounting of the two groups of transducers, optionally through an adaptor base 714. Alternatively, the heat-conducting base comprises three or more flat surfaces, shaped and sized to allow the attachment of transducers groups. In some embodiments, for example as shown in FIG. 7I, an angle 740 between two groups of conductors sharing the same heat-conducting base is in a range of 20-180 degrees, for example 30 degrees, 50 degrees, 90 degrees or any intermediate, smaller or larger degrees value.

According to some exemplary embodiments, two or more TECs are attached to a single cooling bath. In some embodiments, for example as shown in FIG. 7K, the water bath 724 comprises two or more surfaces for attachment of TECs, for example TEC 718. In some embodiments, a hot surface of each TEC out of the plurality of TECs, is attached to a single flat surface of the water bath 724. In some embodiments, the number of flat surfaces of the cooling bath is similar to the number of TECs used in the ultrasound applicator.

### Exemplary Vagina Treatment

According to some exemplary embodiments, an ultrasound applicator is introduced at least partly into a vagina, for example to deliver ultrasound energy into the vagina wall. In some embodiments, the delivered ultrasound energy is used for vagina rejuvenation treatments. In some embodiments, the ultrasound energy is delivered to deep tissue layers within the vagina wall.

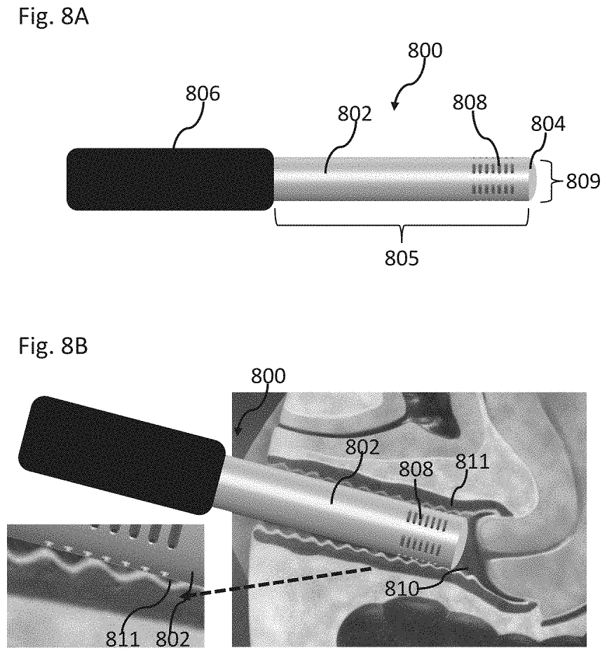

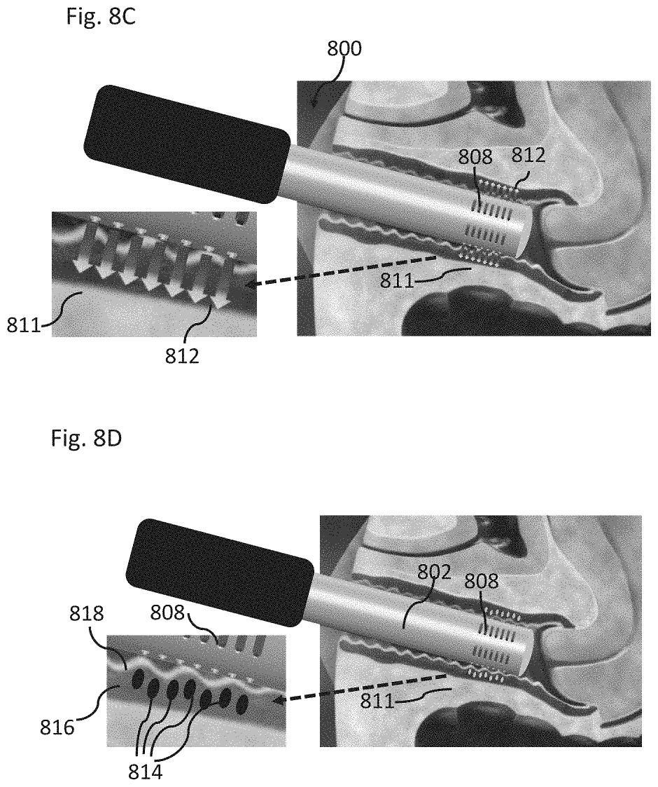

According to some exemplary embodiments, during the delivery of the ultrasound energy, superficial tissue layers of the vagina wall are cooled, for example to reduce the risk of thermal damage. In some embodiments, keeping superficial tissue layers cool allows, for example to provide a prolonged treatment which is optionally more efficient. In some embodiments, the superficial tissues are in contact with the applicator body. Alternatively, the superficial tissues are positioned proximally to the applicator body. Reference is now made to FIGS. 8A-8D, depicting the use of an ultrasound applicator for vagina rejuvenation treatments.

According to some exemplary embodiments, an ultrasound applicator 800 has an elongated applicator body 802, and a gripping member 806 axially connected to the body 802. In some embodiments, the applicator body has a length in a range 4-20 cm, for example 3-12 cm, 5-15 cm, 7-20 cm or any intermediate, smaller or larger range of values. In some embodiments, a width 809 of the applicator body or a portion of the applicator body that is introduced into the vagina is in a range of 1-12 cm, for example 1-5 cm, 2-6 cm, 3-7 cm or any intermediate, smaller or larger range of values.

According to some exemplary embodiments, the ultrasound applicator, for example ultrasound applicator comprises a plurality of ultrasound transducers, which are radially spaced apart on or close to the non-planar surface of the applicator body 802. Alternatively, the ultrasound transducers, for example transducers 808 shown for example in FIG. 8A, are radially and axially spaced apart on or close to the non-planar surface of the applicator body 802. In some embodiments, the radius of curvature of the non-planar surface is smaller than 3 cm, for example 2 cm, 1 cm or any intermediate, smaller or larger value.

According to some exemplary embodiments, the surface of the applicator body 802 or the portion of the applicator body that is introduced into the vagina is smooth, for example not to harm the vagina wall during introduction and/or treatment. In some embodiments, the width of the applicator body is smaller than the width of the vagina. Alternatively, the width of the applicator body is larger than the width of the vagina, for example to ensure contact between the applicator body and the vagina wall.

According to some exemplary embodiments, a cross-section of the applicator body or the portion of the applicator body that is introduced into the vagina is round, oval, ellipsoid, triangular, rectangular or shaped as a polygon.

According to some exemplary embodiments, for example as shown in FIG. 8B, the ultrasound applicator body 802 is introduced at least partly into the vagina 810. In some embodiments, a user holding the applicator 800 by the gripping member advances the applicator body 802 at least partly into the vagina. Optionally, the user rotates the applicator 800, for example to reach a desired alignment between the transducers 808 and a selected target region in the wall of the vagina. In some embodiments, markings and/or a ruler on the surface of the applicator body 802, optionally close to the gripping member 806, provide indications regarding the depth and/or the orientation of the applicator body 802 within the vagina 810. In some embodiments, the ultrasound applicator body is covered prior to the introduction into the vagina. In some embodiments, a friction-reducing material for example a gel is applied on the cover prior to introduction. Alternatively, the gel is applied on the surface of the applicator body prior to introduction into the vagina 810.

According to some exemplary embodiments, the applicator body 802 is introduced into a selected position or to a selected depth within the vagina. In some embodiments, the applicator body 802 is introduced into the vagina to a location that positions the transducers 808 is a close proximity to a treatment target. In some embodiments, the surface of the applicator body 802 is pushed against the vagina wall 811, for example to place the transducers 808 in close proximity to one or more selected targets in vagina wall 811.

According to some exemplary embodiments, when reaching a selected location within the vagina, ultrasonic waves 812 are emitted from at least some of transducers 808 towards the vagina wall 811. In some embodiments, the ultrasonic waves 812 are emitted simultaneously at angular directions larger than 90 degrees, for example 95 degrees, 100 degrees, 180 degrees or any intermediate, smaller or larger value. In some embodiments, the ultrasonic waves are emitted with high frequency values. In some embodiments, the high frequency values are larger than 10 MHz, for example 10.5 MHz, 11 MHz, 12 MHz or any intermediate, smaller or larger value, and intensities of 5-50 W/cm\hat{ }2, for example for example 5-20 W/cm\hat{ }2, 15-30 W/cm\hat{ }2, 20-50 W/cm\hat{ }2, or any intermediate, smaller or larger value. In some embodiments, the high frequency values allow penetration of the ultrasonic waves to tissue layers within the vagina wall, that are close to the internal vaginal wall, for example into the Lamina propria tissue layer.

According to some exemplary embodiments, for example as shown in FIG. 8D, the ultrasonic waves deliver ultrasonic energy to different target regions, for example target regions 814 located in deep tissue layers of the vagina wall, for example in the Lamina propria 816 tissue layer. In some embodiments, the delivered ultrasonic energy heats the target regions. In some embodiments, during the delivery of the ultrasonic energy into the Lamina propria 816, the epithelium layer 818 is unaffected by the thermal energy of the ultrasonic waves. In some embodiments, the applicator body 802 cools the epithelium layer contacting the surface of the applicator body, for example to prevent or minimize thermal damage of the tissue.

According to some exemplary embodiments, a user retracts the applicator 800 out from the vagina while delivering ultrasonic energy at selected locations.

### Exemplary Urethra Wall Treatment

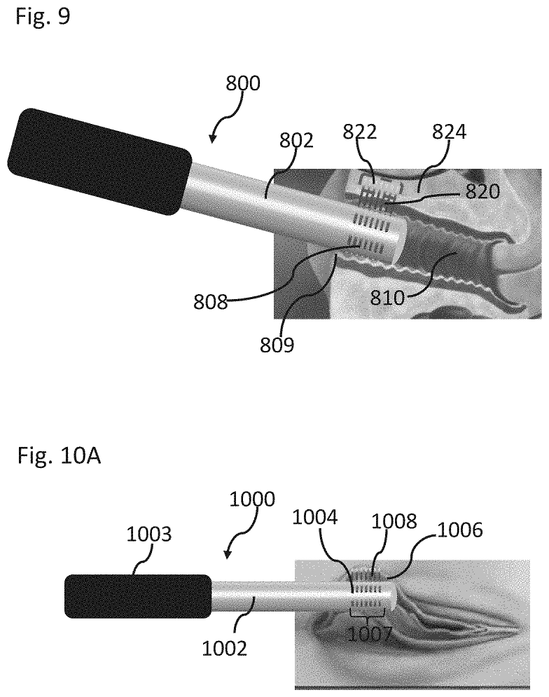

Reference is now made to FIG. 9, depicting a urethra wall treatment, for example when treating urinary incontinence, according to some exemplary embodiments of the invention.

According to some exemplary embodiments, the ultrasound applicator 800 is introduced at least partly into the vagina 810. In some embodiments, the transducers 808 are positioned close to the opening of the vagina at a distance of 5-30 mm from the opening 809, for example in a distance of 5 mm, 10 mm, 30 mm, or any intermediate, smaller or larger value. Optionally, the location of the transducers 808 within the vagina 810 is selected to be the closest to the urethra wall 824. In some embodiments, once in location, ultrasonic waves are emitted towards the urethra wall 824 or towards a tissue volume near the urethra wall, for example to a tissue volume located at a distance of up to 15 mm from the urethra wall. In some embodiments, the ultrasound applicator 800 contacts at least partly the vagina wall during the delivery of ultrasonic waves, optionally while cooling the vagina wall being contacted by the applicator.

According to some exemplary embodiments, the ultrasonic waves are emitted towards the urethra wall 824 and/or towards a tissue volume near the urethra wall, are low frequency ultrasonic waves with a frequency lower than 11 MHz, for example 10 MHz, 9 MHz, 5 MHz or any intermediate, smaller or larger values. In some embodiments, the ultrasonic waves emitted towards the urethra wall have a frequency in a range of 4-10 MHz, for example 5 MHz, 7 MHz, 10 MHz or any intermediate, smaller or lager value. In some embodiments, the emitted ultrasonic waves are kept in a specific range of frequencies, for example to prevent thermal damage in tissues deeper than the urethra wall, for example the urethra itself. In some embodiments, the urethra is filled with cooling fluid, in order to protect it from extra heating.

According to some exemplary embodiments, a catheter is introduced into the urethra. In some embodiments, the catheter is configured to cool the urethra and the urethra wall during the delivery of the ultrasonic waves from the vagina.

### Exemplary Labia Treatment

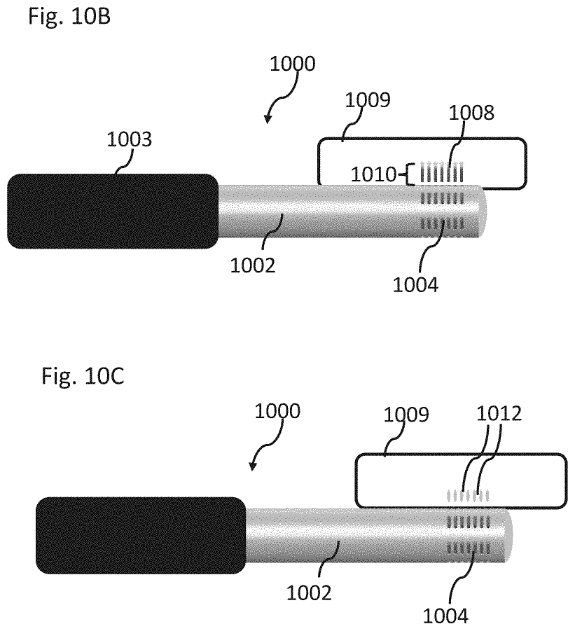

According to some exemplary embodiments, ultrasound energy is used for labia reshaping. In some embodiments, ultrasonic energy is emitted into the major or minor labia tissue, optionally while applying force on the treated tissue. Reference is now made to FIGS. 10A-10C depicting delivery of ultrasound energy into the labia tissue, according to some exemplary embodiments of the invention.

According to some exemplary embodiments, an ultrasound applicator, for example ultrasound applicator 1000 comprises an applicator body 1002, which optionally has a non-planar surface. In some embodiments, the applicator 1000 comprises a plurality of axially spaced apart transducers, for example transducers 1004, positioned near the surface of the applicator body 1002. Additionally or alternatively, the transducers 1004 are radially spaced-apart from each other. In some embodiments, the transducers 1004 are distributed along an axial distance 1007 in a range of 5-50 mm, for example in an axial distance range of 5-15 mm, 10-30 mm, 20-50 mm, or any intermediate, smaller or larger axial distance range.

According to some exemplary embodiments, a portion of the applicator body surface containing some of the ultrasound transducers is pressed against the labia wall 1006 while ultrasound waves 1008 are emitted from at least some of the transducers 1004. In some embodiments, heating tissue layers inside the labia while applying force on the tissue allows reshaping of the labia.

According to some exemplary embodiments, for example as shown in FIG. 10B, ultrasonic waves 1008 are emitted with a frequency in a range of 2-20 MHz, for example 2-10 MHz, 5-10 MHz, 3-15 MHz or any intermediate, smaller or larger range of values, into the Labia 1009. In some embodiments, the ultrasonic waves 1008 penetrate to a depth 1010 of 0.5-5 mm into the Labia 1009, for example into a depth of 0.5 mm, 1 mm, 2 mm, 3 mm, 4 mm, 5 mm or any intermediate, smaller or larger value. In some embodiments, superficial tissue of the labia wall 1009 contacting the applicator is cooled during the treatment.

According to some exemplary embodiments, for example as shown in FIG. 10C, the emitted ultrasonic waves heat target regions 1010, leading to thermal lesions in the target regions 1012.

Exemplary Ultrasound Applicator with Forward Facing Transducers

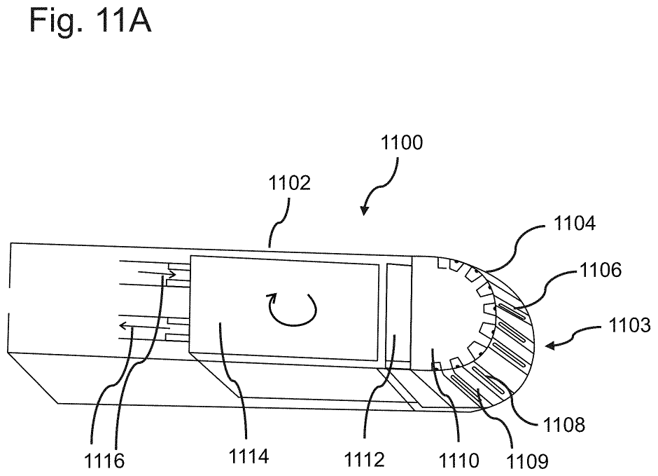

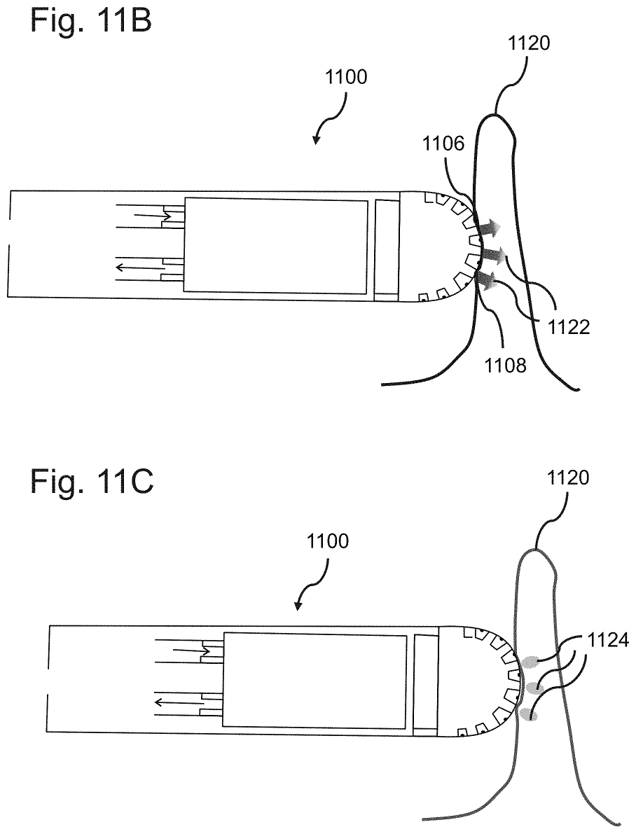

According to some exemplary embodiments, an ultrasound applicator comprises a plurality of spaced-apart forward facing ultrasound transducers. In some embodiments, having forward facing ultrasound transducers allows to better control the pressure applied on the tissue by the transducers. Reference is now made to FIGS. 11A-C, depicting an ultrasound applicator with forward facing ultrasound transducers, according to some exemplary embodiments of the invention.

According to some exemplary embodiments, an ultrasound applicator 1100 has an elongated body with a non-planar surface distal end, for example distal end 1103. In some embodiments, the ultrasound applicator 1100 comprises a plurality of ultrasound transducers, for example 2 transducers, 5 transducers, 10 transducers distributed on the non-planar surface of the distal end 1103. In some embodiments, the transducers are axially and/or radially spaced apart from each other. In some embodiments, the non-planar surface is shaped and sized to be placed in contact with a soft tissue. Optionally, the non-planar surface is smooth, for example to reduce friction with a tissue contacting the surface.

According to some exemplary embodiments, the ultrasound transducers are, for example flat and/or thin transducers, are attached to a base, for example a heat-conducting base. In some embodiments, the base 1110 comprises a plurality of spaced apart protrusions. In some embodiments, a lower surface of each transducer is attached to an upper surface of a base protrusion. In some embodiments, the base 1110 is shaped and sized to conduct heat away from the transducers and/or to cool the transducers by a cooling element within the applicator. In some embodiments, the base 1110 comprises a flat surface shaped and sized to be attached to a cold surface also termed herein as a cold face, of a TEC, for example TEC 1112.

According to some exemplary embodiments, a hot surface of the TEC is attached to a flat surface of a cooling bath, for example a water bath 1114, within an internal lumen of the applicator. In some embodiments, a coolant liquid, for example water circulates between the water bath 1114 and a cooling system in a control unit via tubes 1116. In some embodiments, the transducers on the non-planar surface, are electrically connected by one or more PCBs, for example one or more Flex-PCBs. Optionally, the one or more PCBs electrically connected to the transducers comprise one or more thermistors, for example to measure the temperature of the transducers.

Reference is now made to FIGS. 11B and 11C depicting a treatment of the major and/or minor labia, according to some exemplary embodiments of the invention.

According to some exemplary embodiments, the non-planar surface at the distal end of the applicator 1100 is attached to the surface of the labia, for example the major or minor labia. In some embodiments, ultrasound waves, for example ultrasound waves 1122 are emitted towards the labia 1120. In some embodiments, the emitted ultrasound waves 1122 have a frequency of at least 10 MHz, for example 10 MHz, 11 MHz, 12 MHz or any intermediate, smaller or larger frequency value. In some embodiments, the emitted ultrasound waves 1122 are high frequency ultrasonic waves in a range of 10-13 MHZ, for example 11 MHz, 12 MHz or any intermediate, smaller or larger frequency value.

According to some exemplary embodiments, for example as shown in FIG. 11C, the emitted ultrasonic waves lead into deep issue layers and generate a thermal effect at treatment regions 1124 within the Labia tissue. In some embodiments, the treatment regions reside in the dermis tissue layer of the Labia. In some embodiments, the ultrasonic waves generate the thermal effect in the deep layers of the Labia, while superficial tissue layers of the Labia, for example the epidermis layer are not thermally affected by the ultrasonic waves. In some embodiments, the epidermis is cooled by the surface of the applicators contacting the Labia and/or by the transducers of the applicator that contact the Labia.

### Exemplary Tissue Holder Ultrasound Applicator

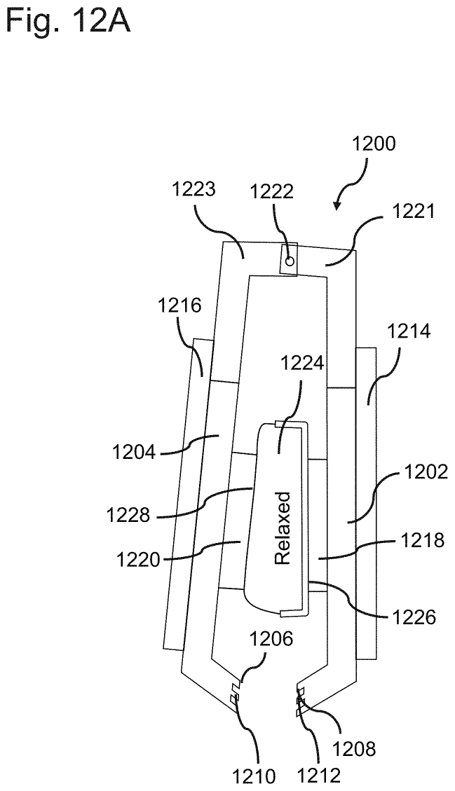

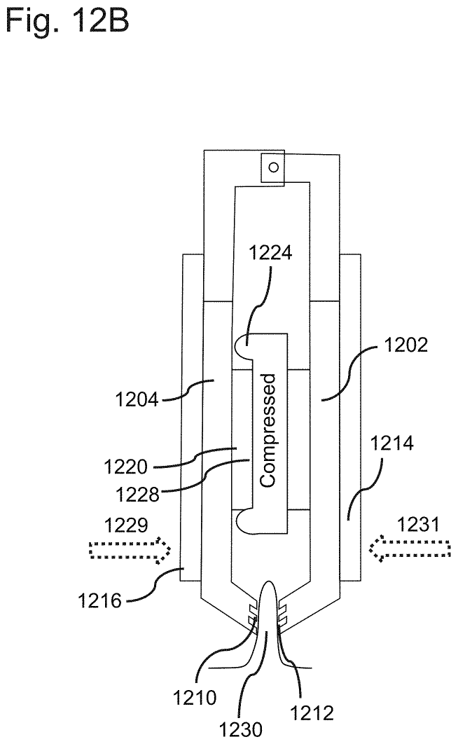

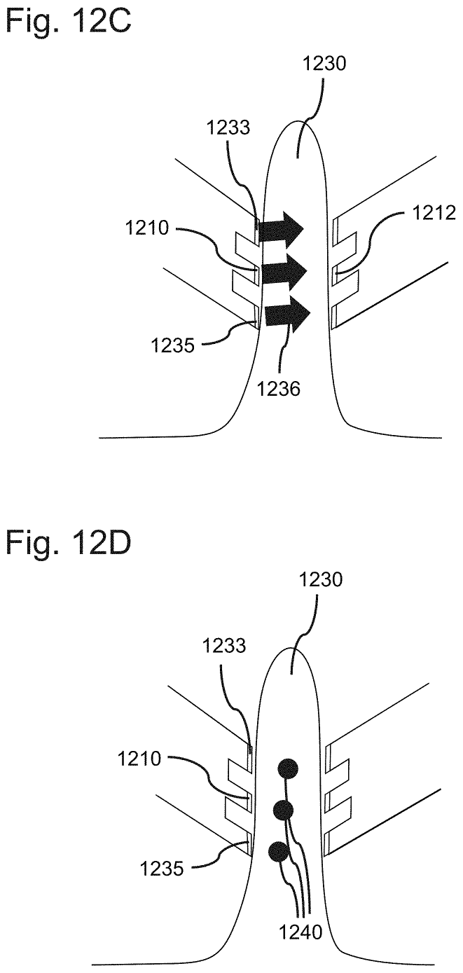

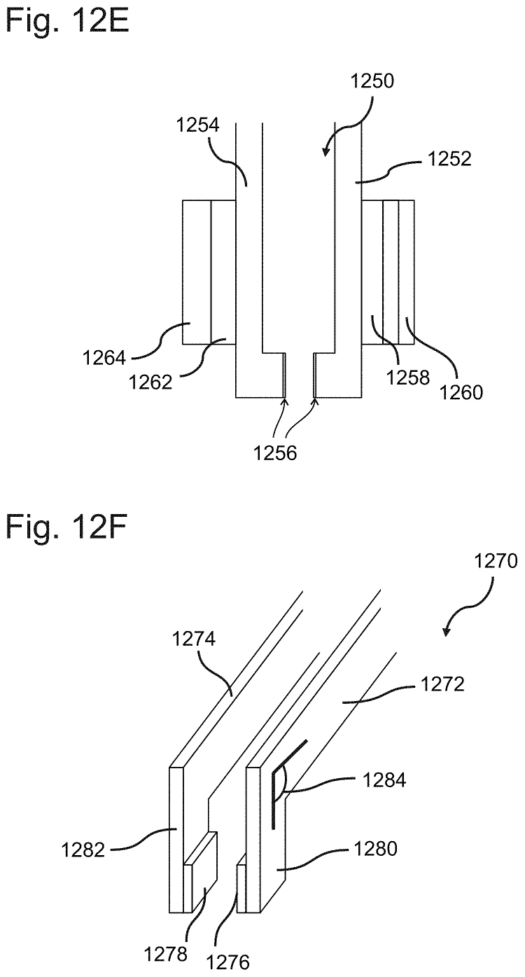

According to some exemplary embodiments, tissue remodeling, for example Labia remodeling is performed by application of ultrasound energy combined with application of pressure on the tissue. Optionally, the thermal effect generated by the ultrasonic energy allows reshaping the tissue to a desired shape by application of force. In some embodiments, an ultrasound applicator is configured to deliver ultrasonic energy, and to apply force on the treated tissue. Reference is now made to FIGS. 12A and 12B, depicting a tissue holder ultrasound applicator, according to some exemplary embodiments of the invention.

According to some exemplary embodiments, an ultrasound applicator, for example applicator 1200, comprises a first surface 1202 and a second opposite surface 1204, optionally configured to move towards each other. In some embodiments, the flat surfaces are made from a thermally isolating material, for example a polymer-based material. In some embodiments, at least one of the surfaces comprises two or more transducers positioned such that movement of the surfaces towards each other holds a tissue portion between the two surfaces and attaches the two or more transducers to the held tissue.

According to some exemplary embodiments, for example as shown in FIG. 12A, the first surface 1202 comprises at least one ultrasound transducer at a distal end 1208, for example transducer 1212. In some embodiments, the transducer 1212, which is optionally a thin and/or a flat transducer, is mounted on an inner face of the first surface facing a second opposite surface 1204. In some embodiments, the second opposite surface 1204 comprises at least one transducer, for example transducer 1210 at a distal end 1206 of the second opposite surface 1204. In some embodiments, the transducer 1210, which is optionally a thin and/or a flat transducer, is mounted on an inner face of the surface 1204 facing the first surface 1202. In some embodiments, the transducers 1210 and 1212 are electrically connected to at least one PCB, which optionally comprises thermistors.

According to some exemplary embodiments, the applicator 1200 comprises two TECs, each TEC is attached to a different surface, for example TEC 1218 is attached to surface 1202 and TEC 1220 is attached to the surface 1204. Optionally, the TECs are attached to an inner face of the surface facing the opposite surface. In some embodiments, a cold surface of the TECs is attached to each of the surfaces 1204 and 1202, which are optionally heat-conducting surfaces. In some embodiments, the heat-conducting surfaces are made from aluminum or any other heat conducting material or alloy.

According to some exemplary embodiments, for example as shown in FIG. 12A, the TECs 1220 and 1218 are attached to a central cooling bath, for example water bath 1224 positioned between the first surface 1202 and the second opposite surface 1204. In some embodiments, a hot surface of each TEC is attached to the water bath 1224, which is made at least partly from a heat conducting material. In some embodiments, at least part of the water bath wall, for example elastic wall 1228 contacting TEC 1220, is elastic, for example to allow movement of the second surface 1204 towards the first surface 1202 while pressing the water bath 1224 positioned between the surfaces. In some embodiments, the elastic wall 1228 comprises an elastic membrane.

According to some exemplary embodiments, the first surface 1202 and the second opposite surface 1204 are pivotally connected by a hinge 1222. Optionally, the hinge 1222 connects the proximal sections, for example sections 1221 and 1223 of the two surfaces 1202 and 1204. In some embodiments, two gripping members 1214 and 1216 are attached to the outer face of each of the surfaces 1202 and 1204, respectively. In some embodiments, the gripping members are shaped and sized to allow, for example, a user to hold the applicator 1200 and move the surfaces 1202 and 1204 closer to each other using a single hand or a portion thereof, for example fingers. In some embodiments, the gripping members are made or covered with a thermal isolator, for example to allow manual grabbing of the applicator.

According to some exemplary embodiments, for example as shown in FIG. 12A, in an open state of the applicator 1200, the first surface 1202 and the second opposite surface 1204 are spaced apart, for example to allow insertion of a tissue portion between the two surfaces, for example between the transducers 1210 and 1212. In some embodiments, for example as shown in FIG. 12B, in a closed state of the applicator 1200, the two surfaces 1202 and 1204 are brought closer to each other by applying force in opposite directions 1229 and 1231 on the gripping members 1216 and 1214 respectively. In some embodiments, when moving the surfaces closer to each other the TEC 1220 presses the elastic wall 1228 of the water bath 1224 causing the water bath 1224 to compress.

According to some exemplary embodiments, for example as shown in FIG. 12B, in the closed state, a tissue portion for example a portion of the Labia 1230 is held by the applicator 1200, optionally pushing at least one of the transducers 1212 and/or 1210 against a surface of the Labia 1230. In some embodiments, in a closed state, the TEC 1220 pushes the elastic wall 1228 of the water bath 1224, causing the water bath 1224 to compress.

According to some exemplary embodiments, for example as shown in FIG. 12C, when a portion of the labia is held by the applicator 1200 in a closed state, ultrasonic waves 1236 are emitted into the Labia 1230. In some embodiments, the ultrasonic waves are emitted into a single side of the labia wall, for example by transducer 1235 of a first surface of the applicator. Alternatively, the ultrasonic waves are emitted into both sides of the Labia, for example by transducers positioned on the two opposite surfaces of the applicator 1200, for example transducers 1210 and 1212. In some embodiments, the ultrasonic waves are emitted simultaneously into both sides of the Labia 1230. Alternatively, the ultrasonic waves are emitted alternately into both sides of the Labia 1230. In some embodiments, during the emitting of the ultrasonic waves into the Labia, superficial tissue layers contacting the transducers, for example the epithelium tissue layer, are cooled down, for example to prevent thermal damage in the epithelium.

According to some exemplary embodiments, for example as shown in FIG. 12D, the ultrasonic waves penetrate into deep layers of the Labia 1230, for example into the dermis. In some embodiments, the delivered ultrasonic energy increases the temperature levels in target regions 1240 in the dermis.

According to some exemplary embodiments, a tissue holder ultrasound applicator comprises two separate cooling system on each opposing surface of the first and second surfaces to allow, for example more efficient cooling of the transducers mounted on each surface. Reference is now made to FIG. 12E, depicting a tissue holder ultrasound applicator with a separate cooling system for each surface, according to some exemplary embodiments of the invention.

According to some exemplary embodiments, an applicator 1250 comprises two opposite surfaces 1252 and 1254, with ultrasound transducers 1256 attached to each surface. In some embodiments, a separate TEC is attached to each of the surfaces, for example a cold surface of TEC 1258 is attached to surface 1252 and a cold surface of TEC 1262 is attached to surface 1254. Optionally, the TECs 1258 and 1262 are attached to the outer face of the surfaces, for example as shown in FIG. 12E. Alternatively, the TECs are attached to the inner face of the surfaces. In some embodiments, the surfaces which are optionally made from a heat conducting material, for example aluminum conduct heat and/or cold between the transducers and the cold surface of the TECs.

According to some exemplary embodiments, a hot surface of each of the TECs is attached to a separate water bath. In some embodiments, a hot surface of TEC 1258 is attached to a surface of water bath 1260. Additionally, a hot surface of TEC 1262 is attached to a water bath 1264. In some embodiments, having two separate water baths allows, for example an efficient way to cool the ultrasound transducers on each of the surfaces. In some embodiments, efficient cooling allows for example, to provide prolonged treatment sessions with high frequency ultrasound energy without damaging the tissue contacting the transducers, for example epithelium tissue.

According to some exemplary embodiments, the tissue holder ultrasound applicator is an angular applicator. In some embodiments, an angular holder ultrasound applicator allows a better control of the force applied on the tissue between the surfaces. Reference is now made to FIG. 12F depicting an angular applicator, according to some exemplary embodiments of the invention.

According to some exemplary embodiments, an angular applicator 1270 comprises two opposite surfaces 1272 and 1274, each comprising two or more transducers on a distal section of each surface. In some embodiments, surface 1272 comprises two or more transducers, for example transducer 1276 attached to an inner face of a distal section 1280. In some embodiments, an opposite surface 1274 comprises two or more transducers, for example transducer 1278, attached to an inner face of a distal section 1282. In some embodiments, an angle 1284 between each distal section of each surface comprising the transducers, and the surface is smaller than 180 degrees, for example 100 degrees, 90 degrees, or any intermediate, smaller or larger angle.

### Exemplary Expanding Ultrasound Applicator

According to some exemplary embodiments, ultrasound waves are delivered to a tissue while a force is applied on the tissue. In some embodiments, the thermal effect of the ultrasonic energy on the tissue in combination with the applied force allows, for example an efficient tissue remodeling process. In some embodiments, an expanding applicator allows to control the force applied by the tissue during treatment. In some embodiments, when the ultrasound applicator is placed in a desired position within the vagina, an expansion mechanism expands a portion of the applicator comprising one or more transducers and pushed the transducers against the vagina wall. In some embodiments, the expansion mechanism allows to push the transducers against the vagina wall with a controlled force.

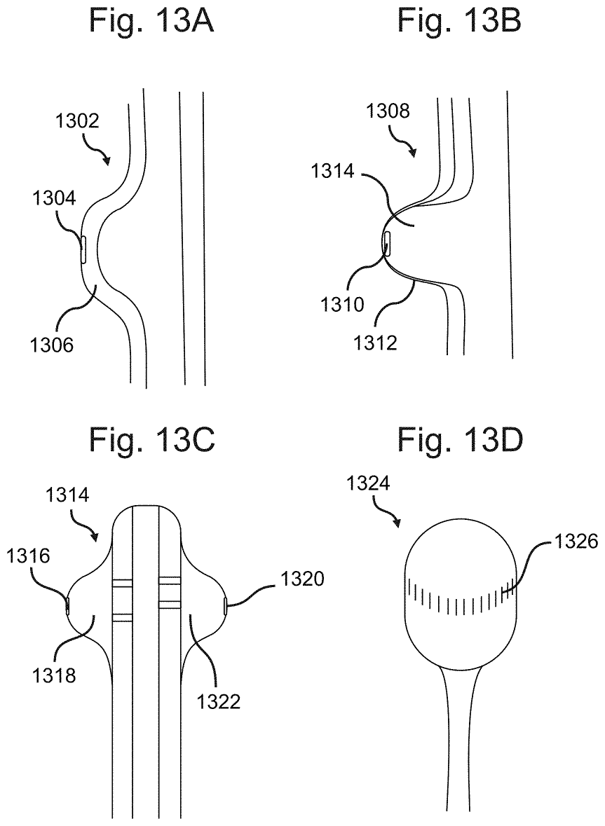

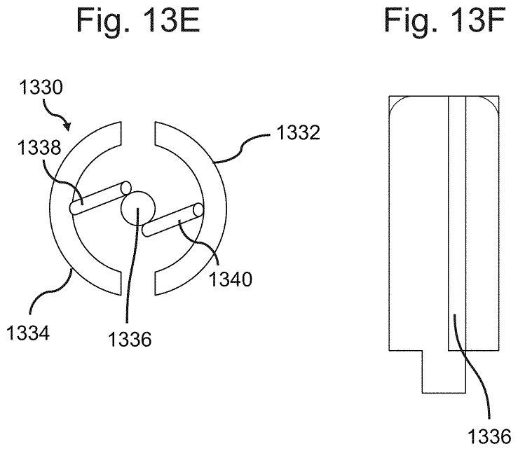

According to some exemplary embodiments, for example as shown in FIG. 13A, an ultrasound applicator 1302 comprises at least one ultrasound transducer mounted on a surface 1306 that is configured to become rigid and/or change a geometrical conformation, for example to push the at least one ultrasound transducer against the tissue, for example against the vagina wall.

According to some exemplary embodiments, for example as show in FIG. 13B, an ultrasound applicator 1308 comprises an expander 1314, for example a balloon, that is configured to selectively push only a selected section of the applicator surface against a tissue. In some embodiments, inflation of balloon 1314 pushes section 1312 comprising at least one transducer, for example transducer 1310 against a tissue, for example against the vagina wall.

According to some exemplary embodiments, for example as shown in FIGS. 13C and 13D, an ultrasound applicator comprises at least one expander configured to push a plurality of radially spaced apart transducers against a tissue, for example against the vagina wall. In some embodiments, for example as shown in FIG. 13C, an ultrasound applicator 1314 comprises two expanders, for example expanders 1318 and 1322, which are configured to expand and push an outer surface of the applicator comprising transducers 1318 and 1320 against a tissue, for example against the vagina wall. Optionally the expanders 1318 and 1322 comprise two separately activated balloons.

In some embodiments, an ultrasound applicator, for example applicator 1324 shown in FIG. 13D comprises a single expander configured to simultaneously push a plurality of radially spaced apart transducers 1325 against a tissue, for example against the vagina wall.

According to some exemplary embodiments, for example as shown in FIG. 13E, an applicator 1330 comprises a body formed from two separable portions, for example portion 1332 and portion 1334. In some embodiments, for example during the delivery of the ultrasound applicator 1330 to a selected position within the vagina, the separable portions are connected to each other. In some embodiments, when reaching a selected position within the vagina, an expanding assembly 1336, optionally comprising a at least one spring, separates the two portions from each other, while pushing each portion against a tissue surface, for example against different parts of the vagina wall. In some embodiments, FIG. 13F is a cross section view of applicator 1130.

### Exemplary Cooling the Transducers

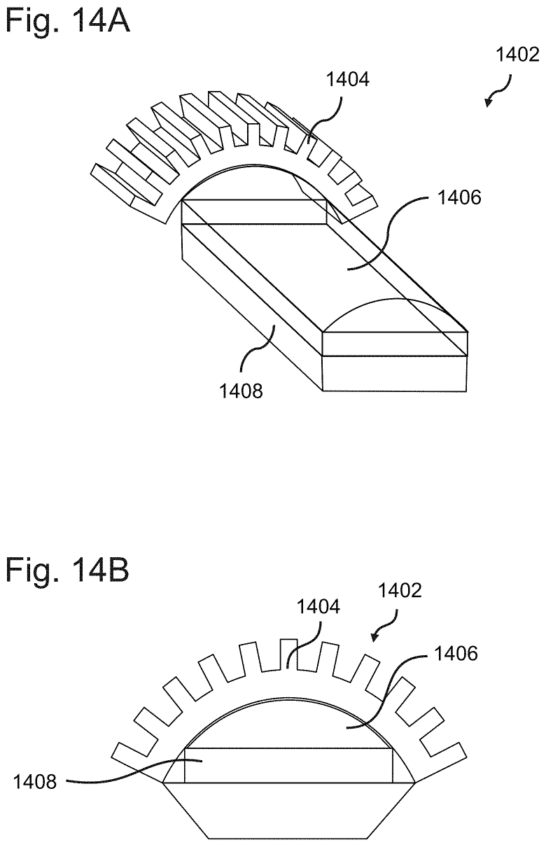

Reference is now made to FIGS. 14A and 14B, depicting cooling transducers of an ultrasound applicators by contacting coolant bath, according to some exemplary embodiments of the invention.

According to some exemplary embodiments, an ultrasound applicator 1402 comprises a plurality of transducers mounted on protrusions of a heat conductor, for example base 1404, which is a heat conducting base. In some embodiments, a coolant liquid bath, for example water bath 1406 contacts a hot surface of TEC 1408, for example to conduct heat from the hot surface of the TEC 1408 into the coolant material, for example cooling liquid inside the water bath 1406. In some embodiments, a cold surface of the TEC 1408 is in contact with a surface of the base 1404, for example to cool the transducers mounted on the protrusions of the base 1404.

Exemplary Ultrasound Applicator with Four TECs

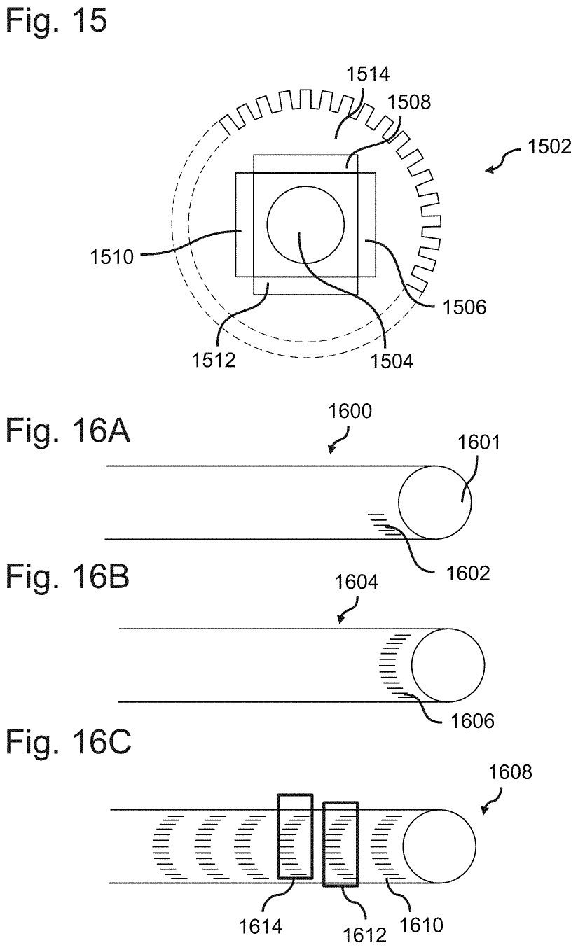

According to some exemplary embodiments, an ultrasound applicator comprises a plurality of cooling elements, for example TECs, to cool a large number of ultrasound transducers and/or to cool a large surface of the applicator body. Reference is now made to FIG. 15, depicting an ultrasound applicator with a plurality of cooling elements, according to some exemplary embodiments of the invention.

According to some exemplary embodiments, an ultrasound applicator comprises a central coolant liquid bath, for example a central water bath 1504. In some embodiments, the central bath is attached to a hot surface of a plurality of cooling elements. In some embodiments, the water bath 1504 is attached to a hot surface of four TECs, for example TECs 1506, 1508, 1510 and 1512. In some embodiments, the cold surface of each TEC is attached to a surface of a single base 1514, which is optionally a heat-conducting base. Alternatively, each TEC is attached to one out of 4 separated base sections, optionally forming together he single base 1514.

### Exemplary Ultrasound Transducers Distribution on an Applicator Surface

According to some exemplary embodiments, ultrasound transducers, for example transducers 1602 shown in FIG. 16A are radially spaced apart in a limited area on the surface of ultrasound applicator 1600, optionally near a distal end 1601 of the applicator.

According to some exemplary embodiments, for example a shown in FIG. 16B, the transducers 1606 are radially distributed on the entire or on a partial section of the perimeter of the ultrasound applicator, optionally close to the distal end of the applicator.

According to some exemplary embodiments, for example as shown in FIG. 16C, the transducers 1610 are distributed on the surface of the applicator in axially spaced apart groups, for example group 1612 and group 1614. In some embodiments, the transducers in each group are radially spaced apart on the entire parameter of the ultrasound applicator. Alternatively, the ultrasound transducers in each group are radially spaced apart on a section of the applicator perimeter. In some embodiments, an axial distance between two groups of radially spaced apart ultrasound transducers is at least 1 cm, for example 2 cm, 4 cm or any intermediate, smaller or larger distance.

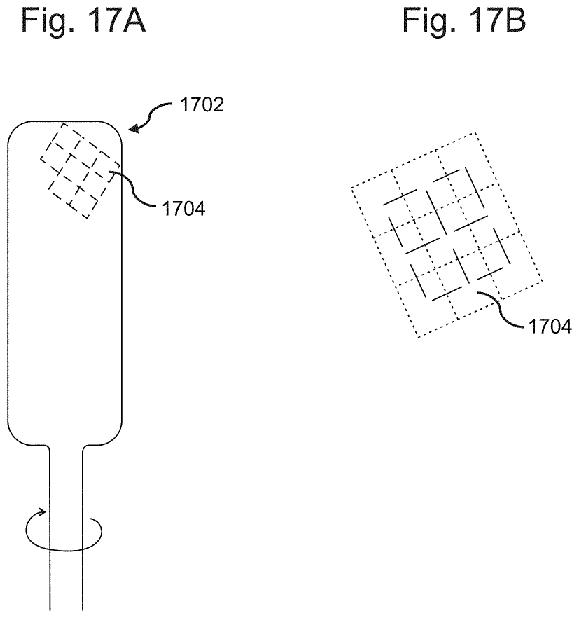

According to some exemplary embodiments, for example as shown in FIGS. 17A and 17B, the transducers, for example transducers 1704 are organized in at least pre-designed pattern on the surface of an ultrasound applicator 1702. In some embodiments, the pre-designed transducers pattern allows, for example to control the shape and/or the size of a thermal lesion or the shape and/or the size of a treated area.

Exemplary Ultrasound Applicator with Internal Transducers

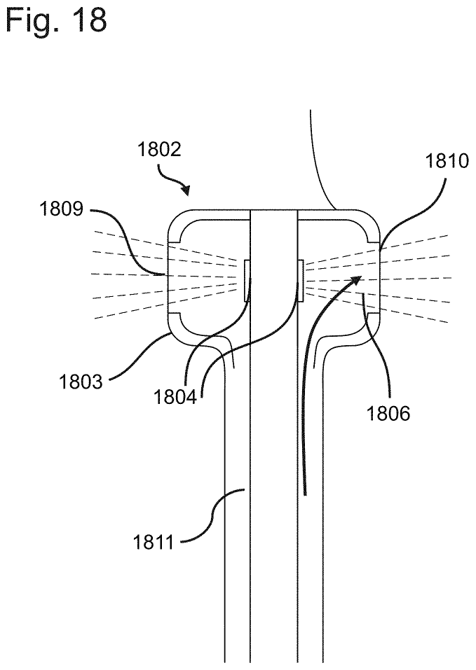

Reference is now made to FIG. 18 depicting an ultrasound applicator with one or more internal transducers, according to some exemplary embodiments of the invention.

According to some exemplary embodiments, an ultrasound applicator 1802 comprises one or more ultrasound transducers, for example transducers 1804, positioned on an internal surface within a lumen of the applicator 1802. In some embodiments, the transducers face a region of the applicator external surface which comprises one or more acoustic windows, for example acoustic windows 1809 and 1810. In some embodiments, the acoustic window, for example acoustic window 1810, allows the delivery of ultrasonic waves emitted from the transducers 1804, to a tissue contacting the external surface of the applicator 1802.

According to some exemplary embodiments, a distal body portion 1803 of the ultrasound applicator 1802 comprising the acoustic windows is wider compared to other parts of the applicator body, for example to allow stretching of the Vagina wall around the acoustic windows. Additionally or alternatively, the wider distal body portion 1803 allows, for example, better contact between the acoustic windows and the tissue.

According to some exemplary embodiments, coolant liquid circulates between the transducers 1804 and the acoustic windows 1809 and 1810, for example to cool the tissue that contacts the distal body portion 1803. Optionally, the coolant liquid 1806 is also ultrasonic waves conductor. In some embodiments, the coolant liquid 1806 comprises water.

Exemplary Ultrasound Applicator with External Transducers

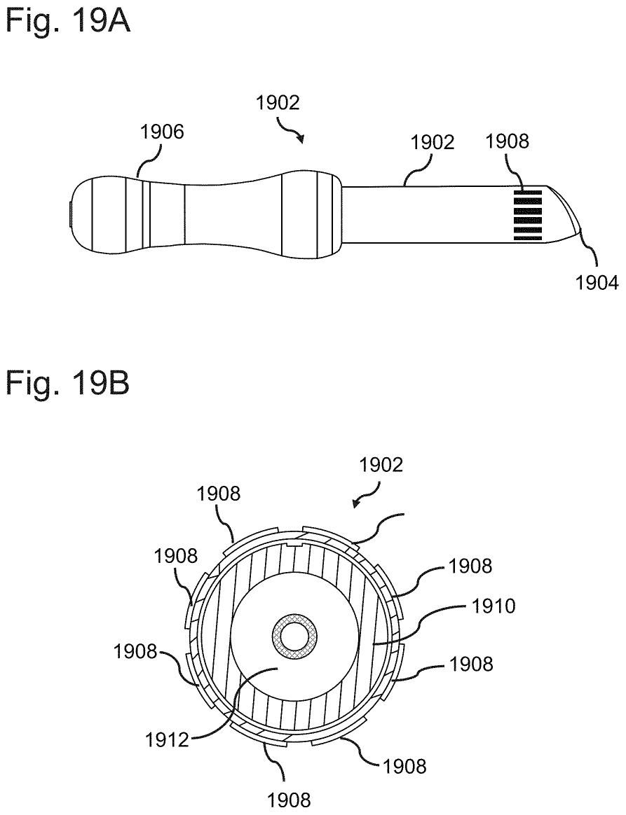

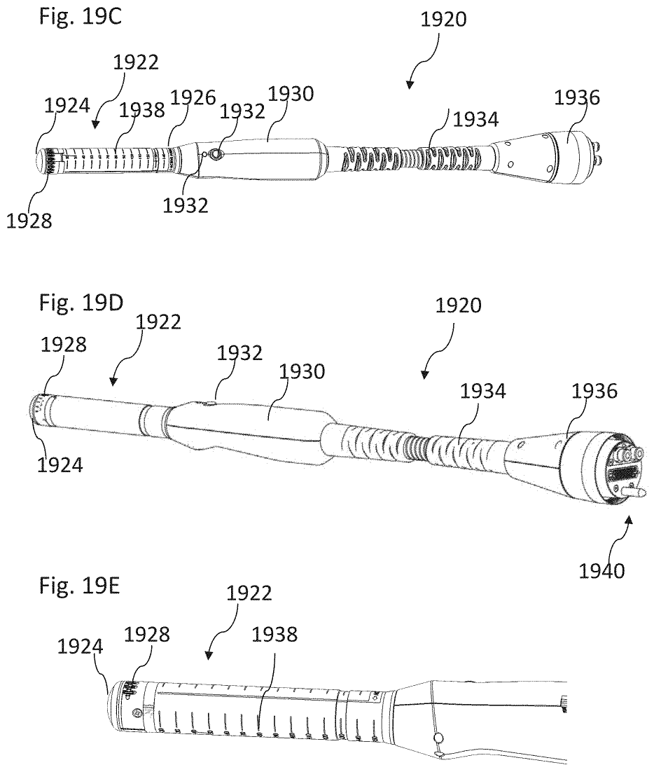

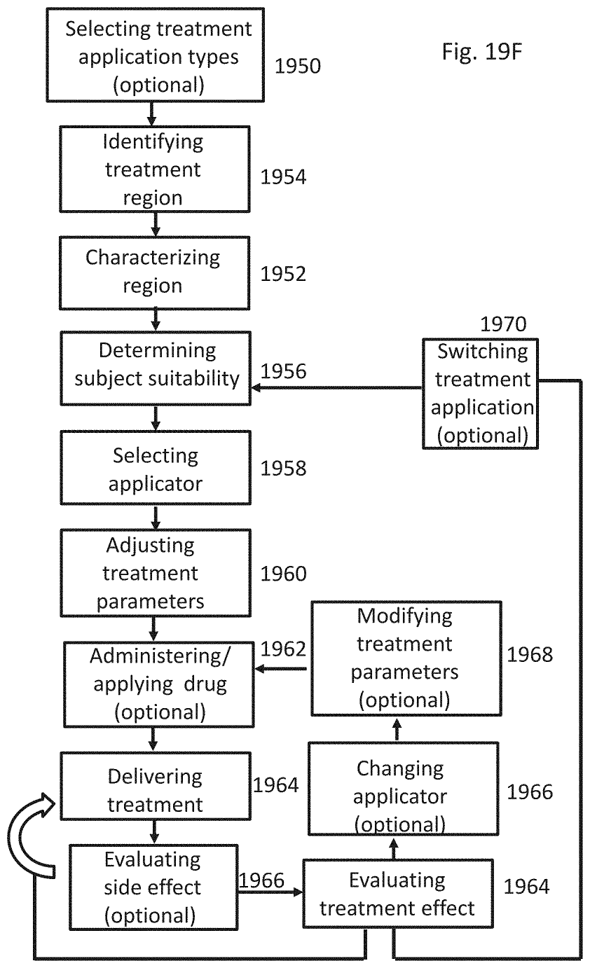

Reference is now made to FIGS. 19A and 19B depicting an ultrasound applicator with external mounted transducers, according to some exemplary embodiments of the invention.

According to some exemplary embodiments, an ultrasound applicator, for example ultrasound applicator 1902 comprises an elongated body, optionally tubular body, for example body 1902, and a gripping member, for example handle 1906. In some embodiments, the handle 1906 is axially connected to the body 1902. In some embodiments, the body 1902 has a distal end 1904, which is optionally an asymmetrical distal end.

According to some exemplary embodiments, for example as shown in FIG. 19B, a plurality of radially spaced-apart ultrasound transducers 1906 are mounted on an external non-planar surface of the body 1902. In some embodiments, a coolant liquid 1910 circulates in an internal lumen of the body 1902 between an internal core 1912 of the ultrasound applicator 1902 and the transducers 1908 or a heat-conducting transducers base. In some embodiments, the coolant liquid 1910, for example water, cools the transducers and/or the outer surface of the ultrasound applicator 1902.

### Exemplary Ultrasound Applicator

Reference is now made to FIGS. 19C-19E depicting an ultrasound applicator, according to some exemplary embodiments of the invention.

According to some exemplary embodiments, an ultrasound applicator, for example applicator 1920 comprises an elongated body, for example body 1922 having a distal end 1924 and a proximal end 1926. In some embodiments, the applicator 1922 comprises a plurality of ultrasound transducers, for example transducers 1928 arranged side-by-side along at least a portion of a circumference of the body 1922. In some embodiments, the transducers have the same axial location on the body 1922 but a different rotation orientation along at least a portion of the circumference of the body 1922. In some embodiments, the body 1922 is at least partly flexible, for example to allow easy navigation within the vagina.

According to some exemplary embodiments, the portion of the circumference comprises an arc in an angle in a range of 10−180° degrees, for example 10-90° degrees, 30-100° degrees, 45-180° degrees or any intermediate, smaller or larger angles range. In some embodiments, for example as shown in FIGS. 19C-19E, the transducers 1928 are arranged side-by-side, optionally in a single line, along the circumference of the body 1922 on an arc smaller than 360° degrees, for example smaller than 270° degrees, smaller than 200° degrees, smaller than 180° degrees or any intermediate, smaller or larger value.

According to some exemplary embodiments, a proximal end 1926 of the body 1922 is coupled to a handle 1920, having one or more gripping members, shaped and sized to be held by a single hand of a user. In some embodiments, the handle 1920 is connected to a tube, for example hollow tube 1934, which comprises one or more electrical wiring and/or coolant liquid tubes traveling within the inner lumen of the flexible tube from the handle 1930 and/or the body 1922 towards connectors 1940. In some embodiments, the connectors 1940 located at a proximal end of the tube 1934, comprise electrical and/or coolant fluid connectors configured to couple the applicator to a control console. Optionally, the tube 1934 is a flexible tube.

According to some exemplary embodiments, the applicator 1920 comprises one or more visual penetration distance indication, for example indications 1938 on the external surface of the body 1922, for example to indicate the penetration distance, for example a penetration depth of the body 1922 into the vagina. In some embodiments, the applicator comprises one or more visual rotation indications, for example to provide indication regarding the rotation of the ultrasound applicator or body 1922 into the vagina.

According to some exemplary embodiments, an ultrasound applicator comprises one or more visual rotation orientation markings on the body, for example body 1922 and/or on the handle 1930. In some embodiments, the visual rotation orientation markings are configured to deliver a visual indication regarding a rotation of the ultrasound applicator within the vagina.

According to some exemplary embodiments, the applicator 1920 comprises one or more activation buttons, for example activation button 1932. In some embodiments, the activation button 1932 is located on the handle 1930, and is configured to allow delivery of ultrasonic energy when the button is pressed. In some embodiments, the applicator 1920 comprises one or more light indicators, for example LED indicator 1932 configured to indicate an activation state of the applicator 1920, for example stand-by state, active state, and/or energy delivery state. Additionally, the applicator 1920 comprises one or more visual or sound indicators, for example one or more LED indicators of, for indicating contact of at least some of the transducers with the tissue, for example with the skin or epithelium.

### Exemplary Treatment

Reference is now made to FIG. 19F depicting a flow of a treatment or a combination of treatment types in the urogenital system, according to some exemplary embodiments of the invention.

According to some exemplary embodiments, one or more treatment application types are selected at block 1950. In some embodiments, the application types comprise one or more vaginal canal tightening, Labia rejuvenation, Stress Urinary Incontinence (SUI), Post-menopausal atrophy, Post radiation dryness and/or symptoms thereof. In some embodiments, for example when two or more applications are selected, a treatment plan is generated.

According to some exemplary embodiments, a treatment region is identified at block 1954. In some embodiments, a treatment region comprises an anatomical location, for example Labia majora, labia minora, vagina canal. Alternatively, the treatment region comprises a region within an anatomical location, for example vagina regions close to the Vulva, vaginal regions close to the Urethra opening, vaginal regions close to the Cervix, and/or one or more selected regions in the Urethra wall.

According to some exemplary embodiments, the treatment region is characterized at block 1952. In some embodiments, the physical properties of the treatment region are characterized per subject, for example the size, diameter, length, surface area and/or the shape of the treatment region is characterized. In some embodiments, the skin folds are characterized in the treatment region, for example presence of skin folds, dimensions and/or depth of skin folds.

According to some exemplary embodiments, the subject suitability to undergo the selected treatment application is determined at block 1956. The subject suitability to undergo the selected treatment application is determined based on one or more of the subject age, subject clinical condition, the medical history of the subject, sensitivity to pain, and/or sensitivity to thermal changes for example increase in heat levels or increase in cold levels.

According to some exemplary embodiments, an ultrasound applicator is selected at block 1958. In some embodiments, the ultrasound applicator is selected according to one or more of the selected treatment application types, the treatment region characterization and the subject suitability to undergo the selected treatment application. In some embodiments, the ultrasound applicator is selected from a plurality of applicators that differ in the number of ultrasound transducers per ultrasound array, number of ultrasound arrays, arrangement of the ultrasound transducers of the applicator, for example ultrasound transducers arranged on an arc in a range of angles between 10-180° degrees, for example 10-50° degrees, 45-90° degrees, 50-180° degrees or any intermediate, smaller or larger range of angles. Alternatively or additionally, the ultrasound applicators differ in the cooling level of the skin, the surface area of the transducers contacting the skin, diameter or maximal width of the applicator and/or length of the applicator. In some embodiments, the applicators differ in their ability to apply mechanical force on the tissue in a timed relationship with the delivery of ultrasonic energy, for example prior to, during and/or after delivery of ultrasonic energy.

According to some exemplary embodiments, the treatment parameters are adjusted at block 1960. In some embodiments, the treatment parameters comprise intensity and/or frequency of ultrasonic energy, duration of a pulse of ultrasonic energy, number of pulses in a train of pulses, per day, per hour, per treatment session and/or per a specific size of surface area. In some embodiments, the treatment parameters are adjusted according to the treatment region characteristics, for example shape and/or size of the treatment region, Additionally or alternatively, the treatment parameters are adjusted according to a depth of a target tissue and/or a distance between the target tissue and other tissues, for example blood vessels and/or nerves. Additionally or alternatively, the treatment parameters are adjusted according to a type of the target tissue, for example fat tissue, lamina propria tissue, fibromuscular layer, adventitia layer or any other tissue type within the target region.

According to some exemplary embodiments, a drug or any other bioactive compound is administered or delivered at block 1962. In some embodiments, the bioactive compound comprises a topical medication, for example a cream or a gel, applied topically to a skin surface contacting at least partly the ultrasound applicator. In some embodiments, topical medication comprises medications that modify thermal sensitivity, for example sensitivity to heat and/or cold, of the skin. In some embodiments, the topical medication comprises medications that reduce sensitivity to pain, for example medications that include lidocaine, prilocaine or any combination thereof. According to some exemplary embodiments, the treatment is delivered to the tissue at block 1964. In some embodiments, the treatment comprises applying mechanical pressure on the tissue in combination with delivery of ultrasonic energy to the tissue. In some embodiments, the ultrasonic energy is delivered to the tissue while cooling the skin. In some embodiments, the treatment is delivered according to the parameter values adjusted at block 1960.

According to some exemplary embodiments, side effects are evaluated at block 1966. In some embodiments, the side effects comprise edema and/or redness in the treated region or regions adjacent to the treated region. Additionally, or alternatively, the side effects comprise pain sensation during or after the treatment. In some embodiments, the side effects are evaluated in a timed relationship with the delivery of the ultrasonic energy, for example during and/or after the delivery of the ultrasonic energy.

According to some exemplary embodiments, the treatment delivery is optionally repeated, at block 1964. In some embodiments, the treatment delivery is repeated a day, a week, a month or any intermediate, smaller or larger time period from the previous treatment delivery. In some embodiments, the treatment delivery is repeated once, twice, 3 times, 5 times, 7 times, 10 times or any smaller or larger number of times. In some embodiments, the treatment delivery is repeated until reaching a desired treatment effect, for example as evaluated at block 1964.

According to some exemplary embodiments, the treatment effect is evaluated at block 1964. In some embodiments, the treatment effect is evaluated at least 10 hours, for example at least 12 hours, at least 24 hours, at least 3 days, at least a week, at least a month or any intermediate, shorter or longer time duration, following the delivery of the ultrasonic energy. In some embodiments, the time duration after which the treatment is evaluated is set according to the selected treatment type, the treatment region and/or the treatment parameters.

According to some exemplary embodiments, if the evaluated treatment effect is not according to a desired effect, then the treatment delivery is repeated. Alternatively, an ultrasound applicator is changed at block 1966 and/or at least some of the treatment parameter values are modified at block 1968.

According to some exemplary embodiments, if the evaluated treatment effect is a desired effect, then the treatment application is optionally switched to a different clinical application at block 1970. In some embodiments, when switching to a different clinical application, the treatment target region is switched.

### Experiment

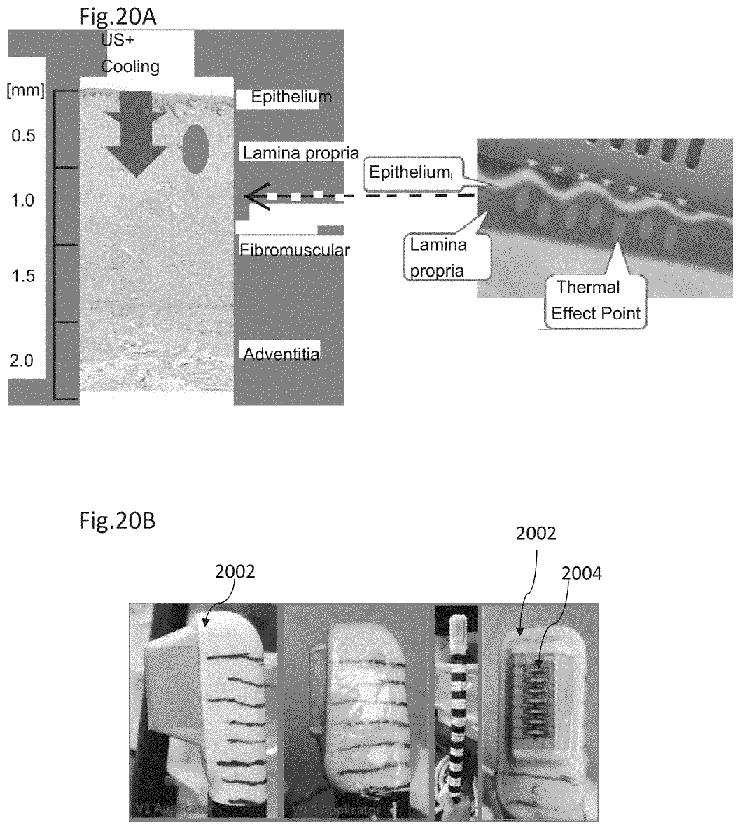

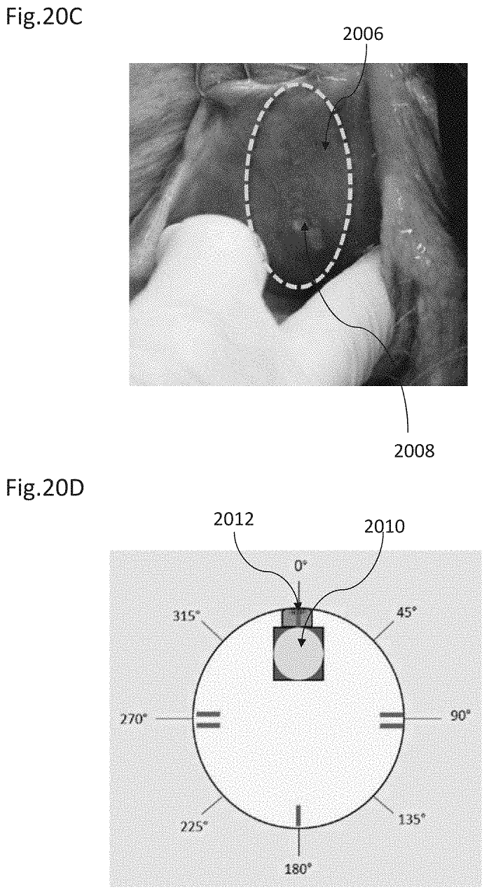

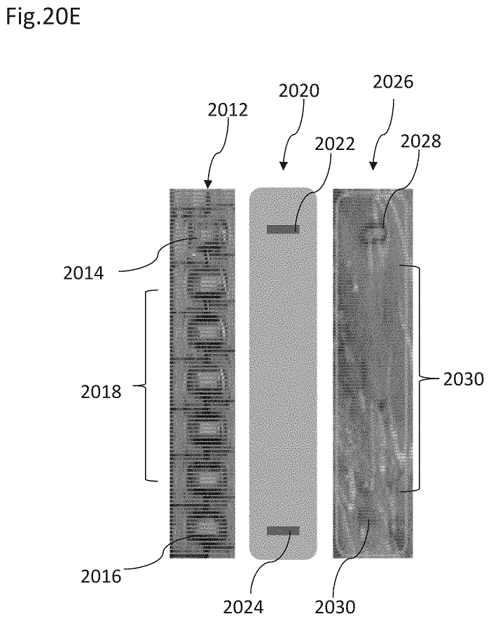

Two experiments were performed in pig animals. In both experiments, and in a treatment according to some exemplary embodiments of the invention, for example as shown in FIG. 20A, ultrasonic energy is delivered by one or more ultrasound transducers placed in contact with the skin, to deep tissue layers, while cooling the skin, for example the epithelium layer. In the experiment and in some embodiments, the skin contacting the transducers was cooled. Alternatively or additionally, skin regions between or adjacent the transducers are cooled.

In the first experiment, and in some embodiments, for example as shown in FIG. 20B, an ultrasound applicator, for example applicator 2002 having a plurality of ultrasound transducers 2004 arranged in a single row, were used. In the experiment and in some embodiments, for example as shown in FIG. 20C, a distal end of the ultrasound applicator comprising the plurality of ultrasound transducers is introduced into the vagina, for example a pig vagina 2006 in the experiment.

In the experiment and in some embodiments, for example as shown in FIGS. 20C and 20D, the ultrasound transducers are placed in contact with the skin. In some embodiments, one or more sensors are configured to sense a contact of one or more of the transducers with the skin, for example by sensing electrical properties or electrical changes, for example changes in impedance and/or conductivity. Alternatively or additionally, contact is sensed based on temperature levels or changes in temperature levels, for example based on signals from one or more thermistors. In some embodiments, the ultrasound applicator comprises one or more thermistors. In some embodiments, the thermistors are placed near at least some of the ultrasound transducers or between adjacent ultrasound transducers. In some embodiments, the thermistors are configured to sense changes in temperature levels of the ultrasound transducers and/or the skin contacting the transducers or the applicator.

In the experiment and in some embodiments, the transducers are placed in contact with the skin at a selected region of the vagina during the application of the ultrasonic energy, for example to treat a selected tissue region in the vagina. In the experiment and in some embodiments, once the selected tissue region was treated, the applicator is rotated at a selected angle for example, in an angle of about 45° degrees or any smaller or larger angle to treat a different tissue region. In the experiment and in some embodiments, the applicator was rotated several times, each time in a degree of about 45° degrees, to cover a complete inner circumference of the vagina. In the experiment and in some embodiments, the applicator is rotated in a selected angle between different tissue regions have a similar axial location. Alternatively and additionally, the applicator is rotated and axially advanced to treat regions that have different circumferential and axial positions.

Reference is now made to FIG. 20E depicting the effect of high and low operation of ultrasound transducers in the experiment and in some embodiments of the invention.

In the experiment and in some embodiments of the invention, the ultrasound transducers of the same applicator, that are placed in contact with the skin are operated at different operation levels, for example different intensities, different frequencies and/or for different time periods. In the experiment and in some embodiments, for example as shown in FIG. 20E, some of the transducers of the transducers array 2012 were operated in high operation levels, for example peripheral transducers 2014 and 2016, while other transducers, for example transducers 2018 were operated in low operation levels. In the experiment and in some embodiments, operating the transducers in high operation levels allowed, for example, to form peripheral marking 2022 and 2024 in 2020, which are optionally burns in the skin, as shown by peripheral markings 2028 and 2030 in the skin 2026. The peripheral markings were used to mark a perimeter of the transducers array 2012, while the transducers 2018 were operated in levels selected to deliver a desired therapeutical effect to the region 2030, defined between the burn markings on the skin 2026.

Additionally, the burn markings on the skin, allowed, for example, to monitor the treated and untreated regions on the skin, optionally by inserting an optic sensor, for example, a camera into the vagina.

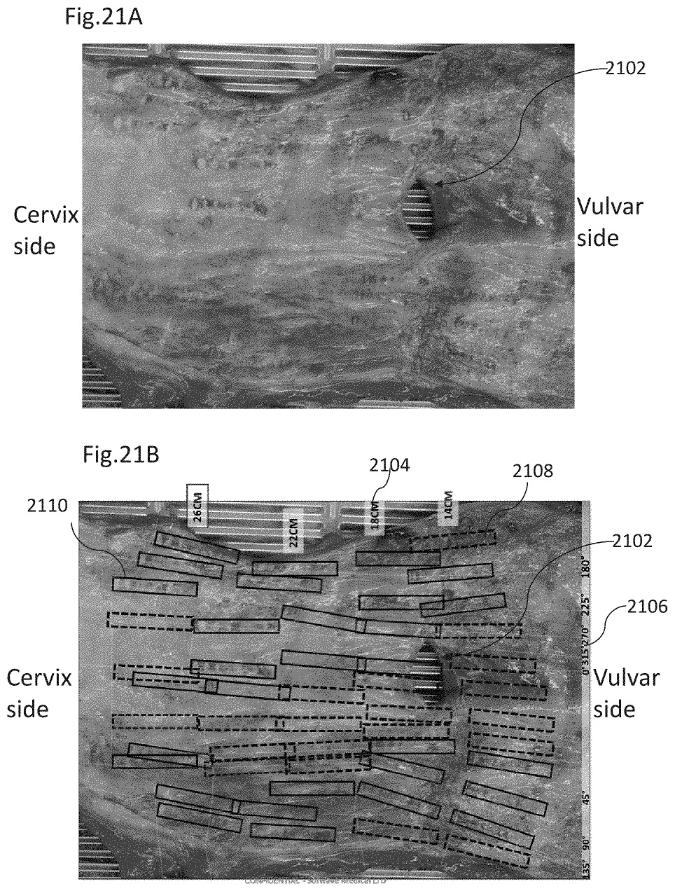

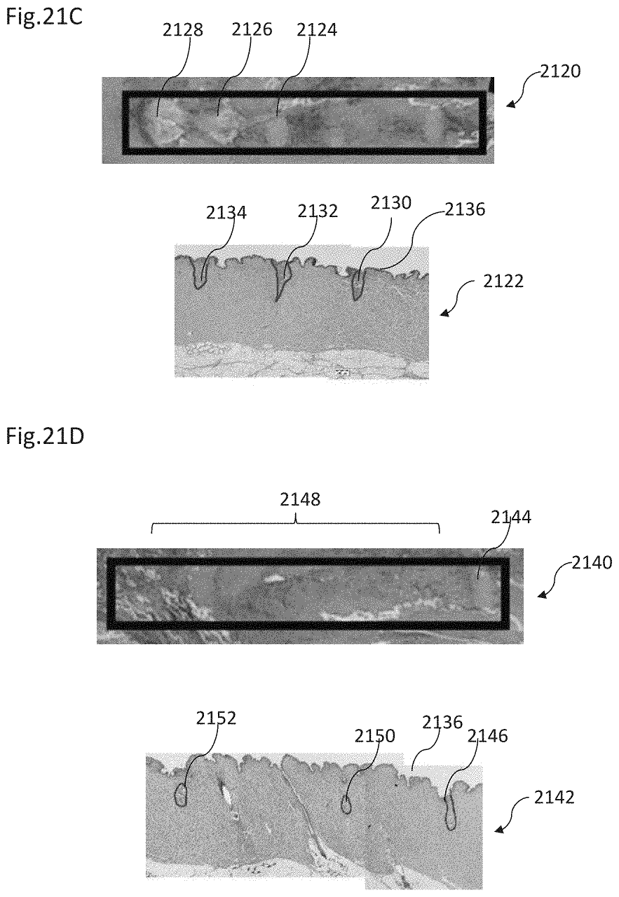

Reference is now made to FIGS. 21A and 21B, areas treated on the vagina skin during the experiment. In the experiment, as shown in FIG. 21A, the treated animal was sacrificed and the vagina skin which includes the urethra opening was removed for further analysis. In the analysis, shown in FIG. 21B, areas where burns are seen on the skin, for example burn area 2110, are marked by solid line rectangles, and areas where minimal or almost no burns are evident, for example area 2108 are marked by dashed line rectangles. The location of each area on the vagina skin is marked by a distance indication related to the distance of the area from the vulvar, for example a distance indication 2104. Additionally, the location of each area on the vagina skin is marked by an angle indication, for example angle indication 2106, related to a rotation angle of the area within the vagina.

Reference is now made to FIG. 21C, depicting areas on the skin showing burn markings on the vagina skin and in a cross-section histochemical staining of the tissue. In the experiment, regions, for example region 2120 showed multiple burning signs, for example burns 2124, 2126 and 2128. These regions correlate with histochemical cross-section staining 2122 showing treated tissue regions 2130, 2132 and 2134 that start from the epithelium layer 2136.

Reference is now made to FIG. 21D, depicting areas on the skin showing some burn markings and areas without burns on the vagina skin and in a cross-section histochemical staining of the tissue. In the experiment, regions that include minimal burn signs, large areas without burns, for example region 2140 indicate regions with potential effect in deep layers of the tissue without damaging the skin. For example, in region 2140, burn 2144 on the skin indicate a damage area starting from the epithelium, for example damage area shown in the histochemical staining 2142. In addition, adjacent areas that do not show any marking, burn or damage on the skin, for example area 2148, indicate a potential effect on deeper areas in the skin, for example on the lamina propria area without causing damage to the epithelium layer 2136, for example in deeper areas 2150 and 2152.

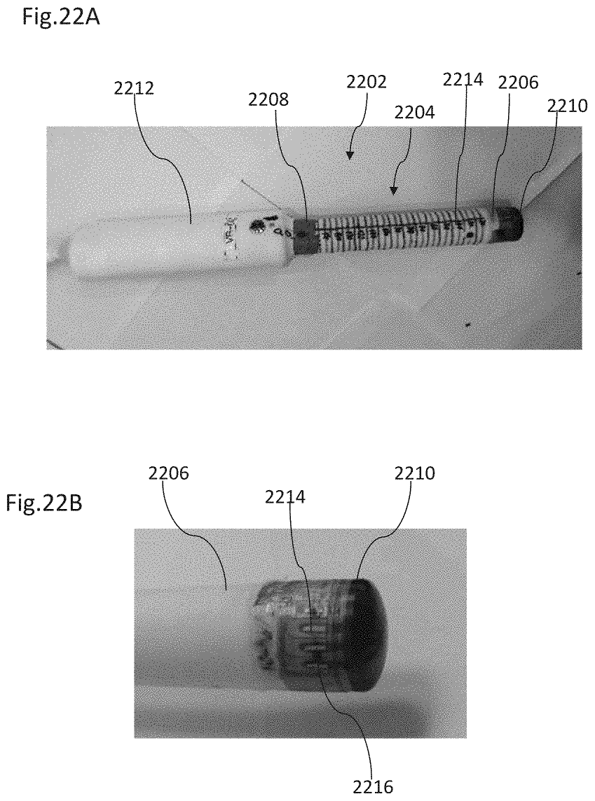

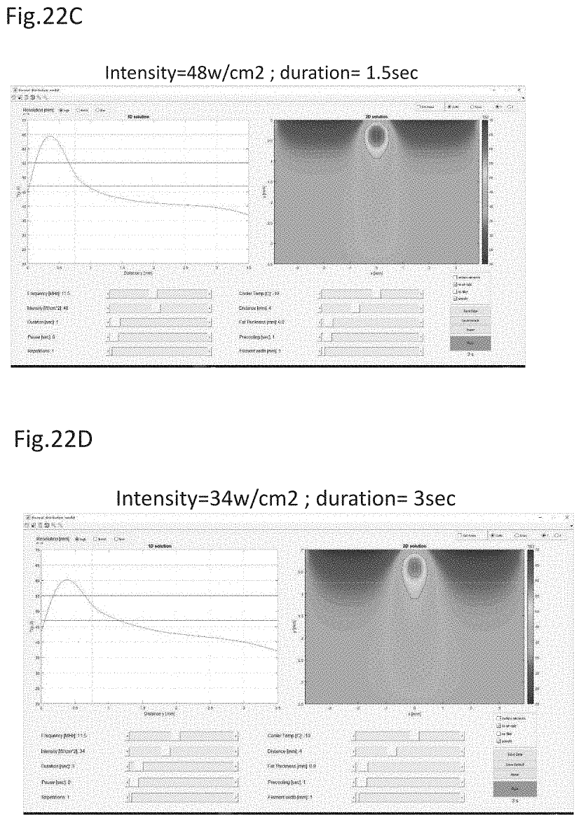

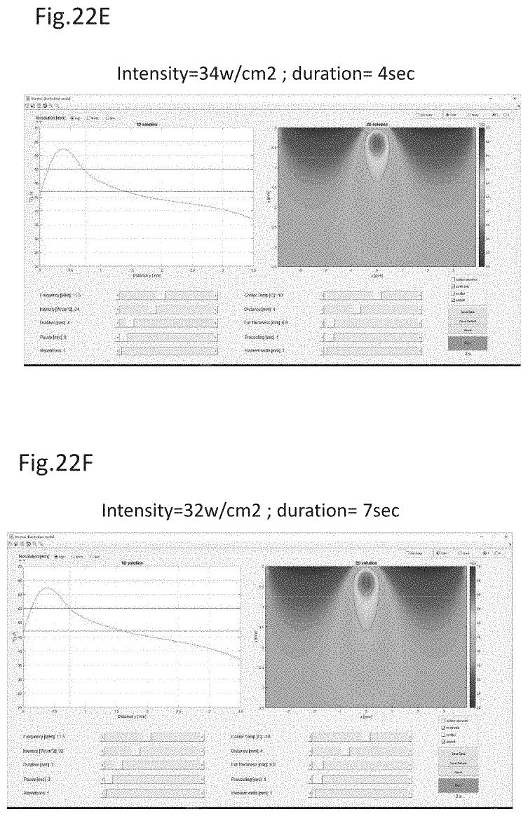

Reference is now made to FIGS. 22A and 22B depicting an ultrasound applicator having a tubular body and comprising a plurality of ultrasound transducers arranged along a curved surface of the elongated body, used in a second experiment and in some embodiments of the invention.

In a second experiment, and in some embodiments of the invention, an applicator having an elongated body 2204 shaped and sized to be introducible into a vagina, has a distal end 2206 and a proximal end 2208. The elongated body 2204 is a tubular, and the applicator 2202 comprises an array 2210 of ultrasound transducers, for example transducers 2214 and 2216 arranged along a curved surface of the applicator body 2204 at the distal end 2206 of the applicator body 2204. In the experiment and in some embodiments, the proximal end 2208 of the tubular body 2204 is connected to a handle 2212, shaped and sized to be held by a hand of a user. Additionally, the tubular body includes one or more visual markings, for example markings 2214 of on the external surface of the body 2204, for example to provide a visual indication related to the insertion depth of the body 2204 into a vagina of a female subject. The applicator used in the second experiment and in some embodiments is similar to applicator 400 shown in FIG. 4A. In applicator 2202 the ultrasound transducers are arranged only on a portion of a perimeter of the body, for example on an arc in a range of 0-180° degrees, for example on an arc of 45° degrees, on an arc of 90° degrees, on an arc of 135° degrees or any intermediate, smaller or larger arc angle.

In the experiment and in some embodiments, simulation is performed prior to treatment, for example to adjust at least of the treatment parameters, for example applied energy intensity, applied energy frequency, applied energy duration, applied cooling levels and/or applied cooling duration. In some embodiments, the treatment parameters are adjusted, for example personalized, according to clinical and/or anatomical condition of a specific subject.

In the experiment and in some embodiments of the invention, simulation is performed prior to treatment, for example as shown in FIGS. 22C-22F. In the experiment, simulation is performed in order to simulate the thermal effect, for example the depth of the thermal effect, on the tissue, for example when applying ultrasonic energy having an intensity of 48w/cm² for a time duration of 1.5 sec (FIG. 22C), when applying ultrasonic energy having an intensity of 34w/cm² for a time duration of 3 sec (FIG. 22D), when applying ultrasonic energy having an intensity of 34w/cm² for a time duration of 4 sec (FIG. 22E), and when applying ultrasonic energy having an intensity of 32w/cm² for a time duration of 7 sec (FIG. 22F).

In the experiment, and in some embodiments of the invention, the ultrasonic energy was applied with the following parameters in the table below while keeping a cooling temperature of about −10° Celsius:

| Duration (sec) | Intensity range [w/cm²] |
|---|---|
| 2 | [36-56] |
| 4 | [32-44] |
| 5 | [28-44] |
| 8 | [28-42] |

Ultrasonic Energy Parameter Values Used in the Second Experiment

Following the experiment, the animal was sacrificed and histochemical analysis was performed on the vagina tissue, for example to identify treatment effect per a specific set or treatment parameters.

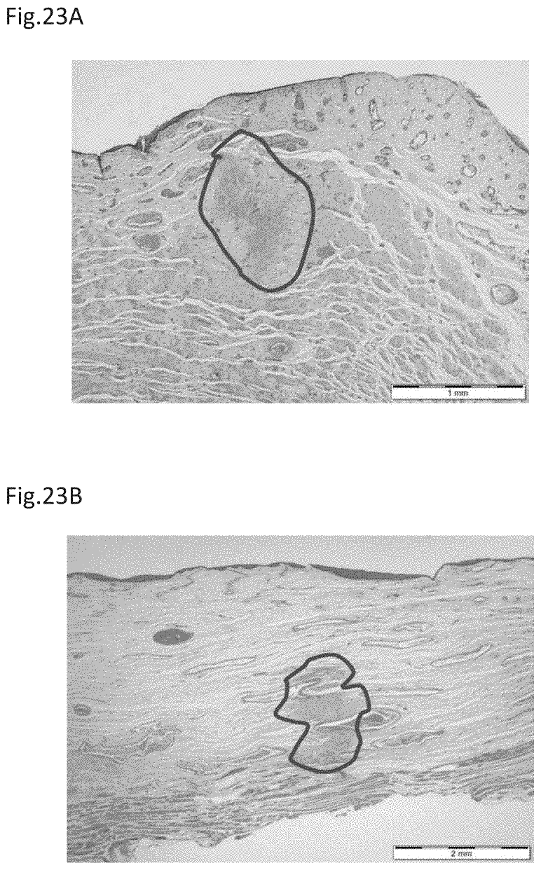

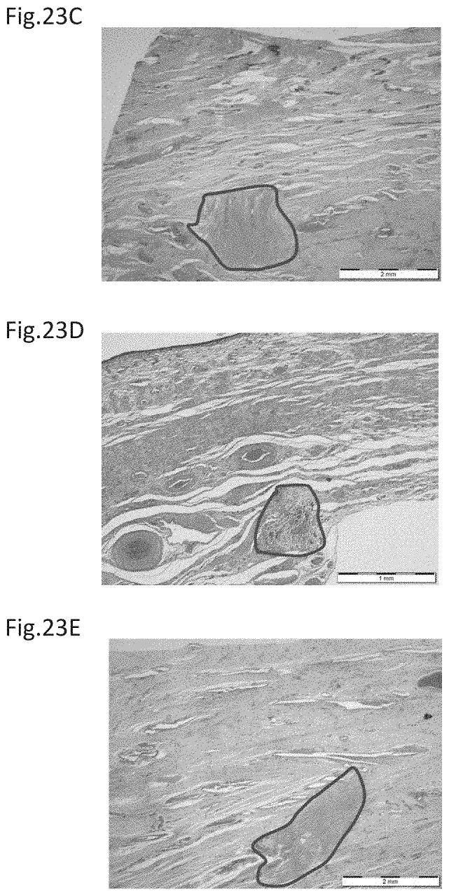

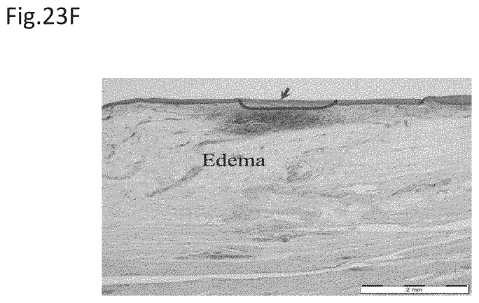

In the experiment and in some embodiments of the invention, an isolated damage to the Lamina propria without evident damage to the epithelium was shown when using intensity levels of 44-48 w/cm² for 2 seconds, as shown in FIG. 23A, and when using intensity levels of 38-40 w/cm² for 8 seconds, as shown in FIG. 23B. In the histochemical images, a perimeter of the treated area is defined by the drawn line.

In the experiment and in some embodiments of the invention, an isolated damage to the Fibromuscular layer without evident damage to the epithelium was shown when using intensity levels of 40-44 w/cm² for 4 seconds as shown in FIG. 23C, when using intensity levels of 48-54 w/cm² for 2 seconds as shown in FIG. 23D, and when using intensity levels of 40-42 w/cm² for 5 seconds as shown in FIG. 23E.

In the experiment, when higher intensities were used for longer time periods, damage to the epithelium layer was observed, for example when using intensity level of 56 w/cm² for 2 seconds, as shown in FIG. 23F, or when using intensity level of 44 w/cm² for 8 seconds. One possible explanation for the epithelium damage is that in these operation parameters, the cooling applied on the epithelium by the applicator is not sufficient to prevent the damage to the epithelium due to the applied ultrasonic energy.

It is expected that during the life of a patent maturing from this application many relevant ultrasound transducers will be developed; the scope of the term ultrasound transducer is intended to include all such new technologies a priori.

As used herein with reference to quantity or value, the term “about” means “within ±10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

1. A system for delivery of ultrasonic waves to a tissue volume, comprising:
an ultrasound applicator shaped and sized to be at least partially inserted into a vagina, comprising:
a body with a non-planar surface, wherein said body is shaped and sized to be at least partly introduced into a vagina;
a plurality of ultrasound transducers distributed on said non-planar surface of said ultrasound applicator body, wherein at least some of said plurality of ultrasound transducers are configured to contact a surface of a wall of said vagina during the generation of ultrasonic waves;
at least one cooler configured to apply cooling to prevent overheating of said vagina wall surface being contacted by said transducers;
a control unit, comprising:
a control circuitry electrically connected to said ultrasonic transducers, wherein said control circuitry signals at least some of said ultrasound transducers to simultaneously generate ultrasonic waves to heat said tissue volume.

2. A system according to claim 1, wherein said non-planar surface has a radius of curvature smaller than 5 cm.

3. A system according to claim 1, wherein said plurality of ultrasound transducers are axially distributed on at least a portion said non-planar surface.

4. A system according to claim 1, wherein said plurality of transducers are circumferentially distributed on at least a portion of said non-planar surface.

5. A system according to claim 1, wherein said body is a tubular body.

6. (canceled)

7. A system according to claim 1, wherein said control circuitry signals at least some of said ultrasound transducers to generate said ultrasonic waves at different angular directions larger than 90 degrees to heat said tissue volume.

8. (canceled)

9. A system according to claim 1, wherein said control circuitry is electrically connected to said cooler, and wherein said control circuitry signals said cooler to apply said cooling prior to and/or during and/or after signaling said at least some of said ultrasound transducers to generate said ultrasonic waves.

10. A system according to claim 1, wherein said at least one cooler is configured to cool said surface of a wall of said vagina being contacted by said transducers through said transducers and/or between said transducers.

11.-15. (canceled)

16. A system according to claim 1, wherein said plurality of ultrasound transducers are thin transducers having a thickness in a range of 0.1-2 mm.

17. A system according to claim 1, wherein said body comprises a plurality of visual depth markings on an external surface of said body, wherein said visual depth markings are configured to deliver a visual indication regarding a penetration depth of the ultrasound applicator into said vagina.

18. A system according to claim 1, comprising a handle coupled to a proximal end of said body, wherein said handle comprises one or more gripping members shaped and sized to allow holding of the handle with a single hand.

19. A system according to claim 18, wherein said ultrasound applicator comprises one or more visual rotation orientation markings on said body and/or on said handle configured to deliver a visual indication regarding a rotation of the ultrasound applicator within the vagina.

20.-21. (canceled)

22. A system according to claim 1, wherein said control circuitry is configured to modify intensity and/or frequency of said ultrasonic waves according to a position of said ultrasound applicator within the vagina and/or according to a relation between said calculated position and a selected target location within the vagina wall.

23. (canceled)

24. A system according to claim 1, wherein said ultrasonic waves generated by said plurality of ultrasound transducers are non converging ultrasonic waves.

25.-29. (canceled)

30. An ultrasound applicator, comprising:
a first surface comprising at least one ultrasound transducer and a second opposite surface coupled to said first surface, wherein said first surface and said second opposite surface move towards each other to clamp a tissue between them;
at least one pressure sensor, configured to sense a pressure applied on said clamped tissue by said first surface and/or said second surface;
wherein said at least one ultrasound transducer emits ultrasonic waves into a tissue volume within said clamped tissue to heat said tissue volume to a temperature larger than 55° C.

31. An ultrasound applicator according to claim 30, comprising a hinge, and wherein said first surface and said second opposite surface are pivotally connected to each other by said hinge.

32. (canceled)

33. A method for delivery of ultrasonic waves to a tissue volume, comprising:
contacting a surface of a vagina wall with a surface of an ultrasound applicator;
simultaneously emitting ultrasonic waves at different angular directions to a tissue volume located in said vagina wall to heat said tissue volume to a temperature larger than 55° C.;
cooling said surface of said vagina wall during said simultaneously emitting.

34. A method according to claim 33, comprises axially moving said surface of said ultrasound applicator to a different axial location within the vagina following said simultaneously emitting.

35. A method according to claim 33, comprising rotating said surface of said ultrasound applicator to a different rotation orientation within said vagina following said simultaneously emitting.

36. A method according to claim 34, comprising modifying intensity and/or frequency of said ultrasonic waves based on said different axial location.

37. A method according to claim 33, comprising positioning said surface of said ultrasound applicator within a liquid sealed introducer prior to said contacting and wherein said cooling comprises cooling said surface of said vagina wall through said liquid sealed introducer.

38. A method according to claim 33, wherein said simultaneously emitting comprises simultaneously emitting ultrasonic waves at angular directions larger than 90 degrees to said tissue volume.

39. A method for reshaping the labia, comprising:
contacting a surface of a labia wall with a surface of an ultrasound applicator;
emitting ultrasonic waves to a tissue volume located within said labia wall to heat said tissue volume to a temperature larger than 55° C., while applying force on said labia wall;
cooling said labia wall surface during said emitting.

40. A method according to claim 39, wherein said contacting comprises contacting a first surface of said labia wall with said surface of an ultrasound applicator and an opposite surface of said labia wall with a different surface of said ultrasound applicator, wherein said emitting comprises emitting ultrasonic waves from said surface and said different surface of said ultrasound applicator to said tissue volume.

41.-45. (canceled)

46. A system for delivery of ultrasonic waves to a tissue volume, comprising:
an ultrasound applicator shaped and sized to be at least partially inserted into a vagina, comprising:
a body with a non-planar surface;
a plurality of ultrasound transducers axially distributed on said non-planar surface of said ultrasound applicator body, wherein said plurality of ultrasound transducers are configured to contact a surface of a wall of said vagina during the generation of ultrasonic waves;
one or more cooling elements configured to apply cooling to prevent overheating of said vagina wall surface being contacted by said transducers, wherein some of said one or more cooling elements are attached to two or more of said plurality of transducers;
a control unit, comprising:
a control circuitry electrically connected to said ultrasonic transducers, wherein said control circuitry signals at least some of said ultrasound transducers to generate ultrasonic waves at different angular directions with parameter values sufficient to deliver ultrasonic energy to said tissue volume.

47. A system according to claim 46, wherein said one or more cooling elements comprises at least one TEC and/or at least one heat-conducting base.

48. (canceled)

49. An ultrasound applicator comprising:
an elongated hollow body having a non-planar surface, wherein said elongated body comprises at least one acoustic window in said non-planar surface;
one or more ultrasound transducers connected to a shaft passing through an inner lumen of said elongated hollow body, wherein said one or more ultrasound transducers are configured to transmit ultrasound waves through said at least one window;
a cooling liquid circulating in said inner lumen between said one or more ultrasound transducers and said at least one window; wherein said circulating cooling liquid reduces temperature levels of said non-planar surface during the activation of said one or more ultrasound transducers.
